(19) United States
(12) Patent Application Publication
(10) Pub. No.: US 2026/0284020 A1
(43) Pub. Date: Sep. 24, 2026

(54) LATS KINASE INHIBITOR TO TREAT RETINAL DEGENERATION

(71) Applicants: Children's Hospital Los Angeles, Los Angeles, CA (US); University of Southern California, Los Angeles, CA (US)

(72) Inventors: Aaron Nagiel, Los Angeles, CA (US); Ksenia Gnedeva, Los Angeles, CA (US); Erik Souverein, Los Angeles, CA (US)

(73) Assignees: Children's Hospital Los Angeles, Los Angeles, CA (US); University of Southern California, Los Angeles, CA (US)

(21) Appl. No.: 19/472,621

(22) PCT Filed: Apr. 4, 2024
(86) PCT No.: PCT/US2024/023146

§ 371 Date: Oct. 3, 2025

Related U.S. Application Data
(60) Provisional application No. 63/456,911, filed on Apr. 4, 2023.

(51) Int. Cl. A61K 31/4375; A61K 9/00; A61P 27/02

(52) U.S. Cl. CPC A61K 31/4375; A61K 9/0048; A61P 27/02; A61K 2123/00

(57) ABSTRACT

Provided herein are methods of treating retinal degeneration, comprising administering a therapeutically effective amount of a LATS kinase inhibitor to a subject with retinal degeneration. In some aspects, the subject has the dry form of age-related macular degeneration (dry AMD) and the method provided herein treats the dry AMD in the subject. In some aspects, the method comprises administering a therapeutically effective amount of TDI-011536 by intravitreal injection to an eye of the subject with dry AMD to treat the dry AMD in the subject. Treating the subject with the disclosed method reduces and/or inhibits retinal degeneration in the subject.

### CROSS REFERENCE TO RELATED APPLICATION

This application claims priority to U.S. Provisional Application No. 63/456,911, filed Apr. 4, 2023, which is incorporated by reference in its entirety.

### FIELD

This disclosure relates to methods of using LATS kinase inhibitors to treat retinal degeneration.

### BACKGROUND

The non-neovascular or dry form of age-related macular degeneration (dry AMD) accounts for nearly ninety percent of all AMD cases and is associated with retinal pigment epithelium (RPE) abnormalities culminating in geographic atrophy (GA) of the RPE and photoreceptors. No definitive treatment to reverse the progression of GA and its associated vision loss exists, representing a large unmet medical need.

### SUMMARY

Disclosed herein is the surprising discovery that administration of a LATS kinase inhibitor treats retinal degeneration in a subject. Accordingly, in some aspects, a method of treating retinal degeneration in a subject is provided. The method comprises administering a therapeutically effective amount of a LATS kinase inhibitor (such as TDI-011536) to a subject with retinal degeneration, such as a subject with dry AMD. In some aspects, the LATS kinase inhibitor is administered to the subject by intravitreal injection to the eye. In some aspects, the LATS kinase inhibitor is administered in no more than five doses (such as a single dose) to treat the subject. In some aspects, the method comprises administering a therapeutically effective amount of TDI-011536 by intravitreal injection to an eye of the subject with dry AMD to treat the dry AMD in the subject. Treating the subject with the disclosed method reduces and/or inhibits retinal degeneration in the subject. In some aspects, treating the retinal degeneration in the subject delays progression (for example, from the early to intermediate stage, or from the intermediate to late stage, of dry AMD) of the retinal degeneration in the subject compared to a control. In some aspects, treating the subject with the method provided herein reduces the number and/or size of drusen under the retina in the subject.

Also provided are in vitro and in vivo methods of identifying an agent for treating retinal degeneration.

The foregoing and other features of this disclosure will become more apparent from the following detailed description of several aspects which proceeds with reference to the accompanying figures.

### BRIEF DESCRIPTION OF THE FIGURES

FIGS. 1A-1G. 1A. Brightfield image (10×) of primary human fetal RPE monolayer showing representative scratch regions measuring 1100 μm in width. 1B. Brightfield images (10×) showing representative quantified regions obtained every 24 hours for 96 hours after scratch. 1C. Wound closure was significantly increased at all time points with LKI compared to control.

FIGS. 2A-2D. 2A. Brightfield image (10×) showing locations of quantified regions at the scratch site, proximal area, and distal area. The original horizontal scratch is seen in the center of the image. 2B. Representative fluorescence image (10×) showing distribution of EdU+ cells 96 hours after scratch with control (left) and LKI (right). 2C. Representative fluorescence image (10×) at a representative scratch region. Within the scratch region, almost all EdU+ cells were also PAX6+. 2D. There was a significantly higher percentage of EdU+ cells within scratch regions (P=0.04) and proximal regions (P<0.0001) with LKI compared to control. There was no significant difference in the percentage of EdU+ cells in distal regions (P=0.14).

FIGS. 3A-3J. 3A. Schematic diagram showing grid pattern of laser spots used to titrate laser intensity to induce damage to the RPE layer in the rabbit eye. Laser photocoagulation lesions were introduced in a 6×6 grid centered on the horizontal visual streak inferior to the optic nerve head. Laser intensity was increased posterior-anterior by increasing the laser pulse duration from 10 ms to 80 ms. The power was kept constant at 170 mW. 3B. Representative fundus photos immediately following laser photocoagulation at 1- and 4-week time points. Low intensity lesions are seen the left-most column while high intensity lesions are seen in the right-most column. 3C. Representative infrared photos immediately following laser photocoagulation at 1- and 4-week time points. Low intensity lesions are seen the left-most column while high intensity lesions are seen in the right-most column. 3D. Representative OCT images showing appearance of various intensity laser spots immediately following laser photocoagulation. 3E. Representative OCT images showing appearance of various intensity laser spots immediately 1 week after laser photocoagulation. 3F. Representative OCT images showing appearance of various intensity laser spots immediately 4 weeks after laser photocoagulation. 3G. Representative laser lesions of different intensities 1 week after laser photocoagulation (20×); sections are stained with hematoxylin and eosin (H&E). 3H. Representative laser lesions of different intensities 4 weeks after laser photocoagulation (20×); sections are stained with H&E. 3I. Representative laser lesion of 40 ms duration and 170 mW power 1 week after laser photocoagulation (20×); sections are stained with H&E. 3J. Representative laser lesion of 40 ms duration and 170 mW power 4 weeks after laser photocoagulation (20×); sections are stained with H&E.

FIGS. 4A-4E. 4A. Representative fundus photos immediately following laser photocoagulation (day 0) and at 1- and 4-week time points after LKI versus control injection. LKI in suspension is visible after 1 week (bottom right). 4B. Representative OCT images following control versus LKI injection at 1- and 4-week time points. Hyper-transmission defects (\hat{ }) were common in control eyes. Shadowing (*) was common in LKI eyes.

FIGS. 5A-5E. A. Representative brightfield and fluorescence images (10×) of laser spots one week after control injection (top) and LKI injection (bottom). Scale bar is 100 μm. B. Representative brightfield and fluorescence images (10×) of laser spots four weeks after control injection (top) and LKI injection (bottom). C. RPE cross-sectional area in was significantly increased in LKI compared to control at 1 week and 4 weeks. D. The percentage of Ki67+ RPE cells was significantly increased in LKI compared to control at 1 week. There was no significant difference in LKI versus control at 4 weeks. E. The percentage of Ki67+ Sox2+ cells was significantly in increased in LKI compared to control at 1 week. There was no significant difference at 4 weeks.

FIGS. 6A-6F. A. Representative brightfield and fluorescence images (10×) of undamaged regions one week after control injection (top) and LKI injection (bottom). Scale bar is 100 μm. B. Representative brightfield and fluorescence images (10×) of undamaged regions 4 weeks after control injection (top) and LKI injection (bottom). C. There were no Ki67+ RPE cells in undamaged regions after 1 or 4 weeks. D. There were no Sox2/Ki67++ cells in undamaged regions after 1 or 4 weeks. E. In laser regions, there was no significant difference in the percentage of Ki67+ cells in the choroid at 1- or 4-week time points. F. In undamaged regions, there was no significant difference in the percentage of Ki67+ cells in the choroid at 1- or 4-week time points.

FIG. 7. An illustration showing that LKI inhibits phosphorylation of Yap/Taz transcriptional cofactors by Lats kinase, reducing degradation of Yap/Taz and promoting translocation to the nucleus, which leads to changes in gene expression.

### DETAILED DESCRIPTION

Provided herein are compositions and methods for treating retinal degeneration, such as dry age-related macular degeneration (AMD). In some examples, the compositions include administration of a therapeutically effective amount of a LATS kinase inhibitor to the eye of a subject with retinal degeneration.

The role of the Hippo Yap signaling pathway in the retina has previously been linked to the pathologic development of proliferative vitreoretinopathy (PVR) and choroidal neovascularization (CNV). PVR is thought to be epithelial-to-mesechymal transition of RPE cells and leads to intractable fibrosis and retinal detachment in patients. The activation of the Hippo pathway via Yap has been shown to be a critical initial step in this process (Zhang, W. & Li, J. EGF Receptor Signaling Modulates YAP Activation and Promotes Experimental Proliferative Vitreoretinopathy. Invest Ophth Vis Sci 63, 24, 2022). Another scenario that has been linked to Yap activation is choroidal neovascularization (CNV), which is the primary cause of vision loss in wet (neovascular) AMD and other disorders. In the experimental mouse model of laser-induced CNV, multiple groups have shown that Yap activation is necessary for the development of pathological angiogenesis (Kim, J. et al. YAP/TAZ regulates sprouting angiogenesis and vascular barrier maturation. J Clin Invest 127, 3441-3461, 2017) by causing proliferation of endothelial cells (Yan, Z. et al. Inhibition of YAP ameliorates choroidal neovascularization via inhibiting endothelial cell proliferation. Mol Vis 24, 83-93, 2017).

Because Lats kinase inhibitors (LKIs) activate Yap signaling, one might expect the application of LKI to the retina to lead to pathologic CNV or PVR and therefore be unsuitable for use in humans. Surprisingly, however, in vitro and in vivo data provided herein show that LKI leads to regenerative proliferation in the retina without these pathologic sequelae. A non-limiting explanation for this surprising result is that, unlike genetic perturbations which lead to constitutive activation of Yap signaling, the small-molecule LKI provides a short-term, controlled burst of RPE and Müller glia proliferation restricted to the site of damage.

Accordingly, LKIs have utility as therapeutic agents for diseases primarily affecting the integrity of the photoreceptor and RPE layers, particularly in the context of short-term (e.g., administration of up to five doses, such as three doses) treatment by intravitreal injection. This is a major feature of dry AMD (one of the leading causes of blindness worldwide), Stargardt disease, Best disease, myopic macular degeneration, and inherited retinal dystrophies (a diverse set of syndromic and non-syndromic conditions caused by over 250 genes). Unlike many current treatment modalities which arm to slow the rate of disease progression, data provided herein shows that LKIs regenerate the cellular architecture of the retina with a limited number of doses of the drug.

### I. Terms

Unless otherwise noted, technical terms are used according to conventional usage. As used herein, the singular forms “a,” “an,” and “the,” refer to both the singular as well as plural, unless the context clearly indicates otherwise. As used herein, the term “comprises” means “includes.” Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: In the context of a reference value, about refers to plus or minus 5% of the reference value. Thus “about 100” refers to 95 to 105.

Acyl: Unless otherwise specified, acyl refers to formyl and to groups of 1, 2, 3, 4, 5, 6, 7 and 8 carbon atoms of a straight, branched, cyclic configuration, saturated, unsaturated or aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo.”

Administer: To provide or give a subject an agent, such as a therapeutic agent (e.g. a small molecule inhibitor of LATS kinase), by any effective route. Exemplary routes of administration include, but are not limited to, topical administration (for example, eye drops) or injection (such as intravitreal or subretinal injection).

“Administration of” and “administering a” compound should be understood to mean providing a compound, a prodrug of a compound, or a pharmaceutical composition as described herein. The compound or composition can be administered by another person to the subject or it can be self-administered by the subject.

Age-related macular degeneration (AMD): A disease that is a major cause of blindness in the United States and other industrialized nations. Most people with AMD have dry AMD (also called atrophic AMD), which progresses in three stages: early, intermediate, and late. Typically, the disease progresses slowly over several years.

Dry AMD is characterized by the appearance of small yellow deposits called drusen, which form under the retina. Early dry AMD patients typically have several small drusen or a few medium-sized drusen. Drusen are extracellular deposits of proteins, lipids, and cellular debris, that are located beneath the retinal pigment epithelium (RPE). The RPE provides nutritional, metabolic, and phagocytic functions for the overlying photoreceptors. At the early stage, there are usually no symptoms or vision loss. At the intermediate stage, patients have either many medium-sized drusen or one or more large drusen. At this stage, many individuals may still be without symptoms, but some may see a blurred spot in the center of their vision. Those with the intermediate stage of dry AMD also may require more light or contrast (sharpness between light and dark) for reading and other tasks. They are also at increased risk of progressing to the late stage of dry AMD or developing wet AMD. At the late stage, there are large areas of atrophic tissue causing central blind spots in the fovea or center of one's vision. Significant vision loss results from dysfunction or death of photoreceptors in the macula in association with late stage of dry AMD (geographic atrophy of the retinal pigment epithelial cells). At the late stage, patents may have difficulty reading or recognizing faces. The late stage of dry AMD is also known as geographic atrophy (GA).

Wet AMD, also known as neovascular AMD, generally causes more rapid and more serious vision loss if left untreated. In wet AMD, patients develop tiny new blood vessels and scar tissue which grow under and into the retina. These blood vessels are prone to leaking and hemorrhage, which, along with scarring of the retina, can cause a loss of vision.

Alkyl: Unless otherwise specified, alkyl is a linear or branched hydrocarbyl. Unless otherwise specified, an unsubstituted alkyl has from 1 to 20 carbon atoms (e.g., 1 to 6 carbon atoms). Examples of alkyl groups include methyl, ethyl, propyl, isopropyl, butyl, s- and t-butyl and the like.

Aryl and heteroaryl: Refer to (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Arylalkyl: Refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. In one aspect, the alkyl group of an arylalkyl or a heteroarylalkyl is an alkyl group of from 1 to 6 carbons. Examples include, e.g., pyridinylmethyl, pyrimidinylethyl and the like.

Carbocycle: Unless otherwise specified, carbocycle is a ring system in which the ring atoms are all carbon but of any oxidation state. Thus (C₃-C₈) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Cell tissue culture: The process by which either prokaryotic or eukaryotic cells are grown under controlled conditions. In practice the term “cell culture” or “tissue culture” has come to refer to the culturing of cells derived from multicellular eukaryotes, especially animal cells, such as mammalian cells. Mammalian cells are grown and maintained at an appropriate temperature and gas mixture (typically, 37° C., 5% CO₂) in a cell incubator. Culture conditions vary widely for each cell type, and variation of conditions for a particular cell type can result in different phenotypes being expressed. Aside from temperature and gas mixture, the most commonly varied factor in culture systems is the growth medium. Recipes for growth media can vary in pH, glucose concentration, growth factors, and the presence of other nutrient components. The growth factors used to supplement media are often derived from animal blood, such as calf serum.

Some cells naturally live without attaching to a surface, such as cells that exist in the bloodstream. Others require a surface, such as most cells derived from solid tissues. Cells grown unattached to a surface are referred to as suspension cultures. Cell grown on a surface (or “adherent” cultures of cells) can be grown, of example, on tissue culture plastic, which may be coated with extracellular matrix components (for example collagen or fibronectin) to increase its adhesion properties and provide other signals needed for growth. A monolayer of cells grown in tissue culture is a layer of cells that is one cell deep and that occupies substantially all of a target surface on which the cells are grown.

Compound: The term “compound”, unless expressly further limited, is intended to include salts of that compound. In some aspects, the term “compound” refers to the compound or a pharmaceutically acceptable salt thereof.

Control: A sample or standard used for comparison with an experimental sample.

Heterocycle: Refers to a cycloalkyl or aryl carbocycle residue in which from one to four carbons is replaced by a heteroatom selected from N, O and S. The nitrogen and sulfur heteroatoms may optionally be oxidized, and the nitrogen heteroatom may optionally be quaternized. Unless otherwise specified, a heterocycle may be non-aromatic (i.e. aliphatic) or aromatic. Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heteroaromatic rings include: furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Examples of heterocyclyl residues additionally include piperazinyl, 2-oxopiperazinyl, 2-oxopiperidinyl, 2-oxo-pyrrolidinyl, 2-oxoazepinyl, azepinyl. 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygens, as well as other heteroatoms. A sulfur heterocycle is a heterocycle containing at least one sulfur in the ring; it may contain additional sulfur, as well as other heteroatoms. Oxygen heteroaryl is a subset of oxygen heterocycle; examples include furan and oxazole. Sulfur heteroaryl is a subset of sulfur heterocycle; examples include thiophene and thiazine. A nitrogen heterocycle is a heterocycle containing at least one nitrogen in the ring; it may contain additional nitrogen, as well as other heteroatoms. Aliphatic nitrogenous heterocycles include piperidine, piperazine, morpholine, pyrrolidine, thiomorpholine, azetidine, azepine, and azepane. Nitrogen heteroaryl is a subset of nitrogen heterocycle; examples include pyridine, pyrrole and thiazole.

Hydrocarbon or hydrocarbyl: Hydrocarbon or hydrocarbyl (as a substituent) includes alkyl, cycloalkyl, polycycloalkyl, alkenyl, alkynyl, aryl and combinations thereof. Examples include cyclopropylmethyl, benzyl, phenethyl, cyclohexylmethyl, camphoryl and naphthylethyl. Hydrocarbon refers to any substituent comprised of hydrogen and carbon as the only elemental constituents, Cycloalkyl is a subset of hydrocarbyl and includes cyclic hydrocarbon groups of from 3 to 8 carbon atoms. Examples of cycloalkyl groups include c-propyl, c-butyl, c-pentyl, norbornyl and the like.

Injectable composition: A pharmaceutically acceptable fluid composition comprising at least one active ingredient, for example, a LATS kinase inhibitor. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

Intraocular administration: Administering agents locally, directly into the eye, for example by delivery into the vitreous or anterior chamber, or subretinally. Indirect intraocular delivery (for example by diffusion through the cornea) is not direct administration into the eye.

Intravitreal administration: Administering agents into the vitreous cavity. The vitreous cavity is the space that occupies most of the volume of the core of the eye with the lens and its suspension system (the zonules) as its anterior border and the retina and its coating as the peripheral border. Intravitreal administration can be accomplished by injection, pumping, or by implants.

Large tumor suppressor kinase (LATS): A serine/threonine kinase that in humans is associated with the Hippo signaling pathway, where it phosphorylates YAP and TAZ to inactivate their function. The protein sequence of LATS is provided as Genbank Accession No. NP_004681.1, which is incorporated by reference herein.

LATS kinase inhibitor (LKI): A small molecule compound that interacts with an reduces the serine/threonine kinase activity of LATS kinase. Non-limiting examples of LATS kinase inhibitors are provided in PCT Pub. No. WO2021158936, which is incorporated by reference herein in its entirety. A specific example of a LATS kinase inhibitor is TDI-011536.

Müller glia: A type of glial cells found in the vertebrate retina. The major role of Müller glia is to provide structural and metabolic support to the neuronal cell types of the retina, including photoreceptors. In non-vertebrates, Müller glia also can serve as a progenitor and proliferate to replace lost neurons and photoreceptors after damage—the core function that is repressed in mammals.

Optionally substituted: Used interchangeably with “unsubstituted or substituted.” The term “substituted” refers to the replacement of one or more hydrogen atoms in a specified group with a specified radical. For example, substituted aryl, heterocyclyl etc. refer to aryl or heterocyclyl wherein one or more H atoms in each residue are replaced with halogen, haloalkyl, alkyl, (C₁-C₈)hydrocarbyl, acyl, alkoxyalkyl, hydroxyloweralkyl, carbonyl, phenyl, heteroaryl, benzenesulfonyl, hydroxy, loweralkoxy, haloalkoxy, oxaalkyl, carboxy, alkoxycarbonyl [i.e. —C(═O)O-alkyl], carboxamido [i.e. —C(═O)NH₂], alkylaminocarbonyl [i.e. —C(═O)NH-alkyl], cyano, acetoxy, nitro, amino, alkylamino, dialkylamino, dialkylaminoalkyl, dialkylaminoalkoxy, heterocyclylalkoxy, arylalkyl, (cycloalkyl)alkyl, heterocyclyl, heterocyclylalkyl, alkylaminoalkyl, heterocyelylaminoalkyl, heterocyclylalkylaminoalkyl, cycloalkylaminoalkyl, cycloalkylalkylaminoalkyl, arylaminoalkyl, and arylalkylaminoalkyl, mercapto, alkylthio, alkylsulfinyl, benzyl, heterocyclyl, phenoxy, benzyloxy, heteroaryloxy, aminosulfonyl, amidino, guanidino, ureido, —SO₂alkyl, —SO₂NH₂, or —SO₂NHalkyl, Preferred substituents are halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy (C₁-C₃)alkyl, hydroxy(C₁-C₃)alkyl, heteroaryl, benzenesulfonyl, (C₁-C₃)alkoxycarbonyl [i.e. —C(═O)O(C₁-C₃)alkyl], carboxamido [i.e. —C(═O)NH₂], (C₁-C₃)alkylaminocarbonyl [i.e. —C(═O)NH—(C₁-C₃)alkyl], (C₁-C₃)alkylamino, di(C₁-C₃)alkylamino, amino(C₁-C₃)alkyl, (C₁-C₃)alkylamino(C₁-C₃)alkyl, (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkylsulfonyl, phenoxy, and benzyloxy.

Oxaalkyl: Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art (see Naming and Indexing of Chemical Substances for Chemical Abstracts, published by the American Chemical Society, 196, but without the restriction of 127(a)), i.e. it refers to compounds in which the oxygen is bonded via a single bond to its adjacent atoms (forming ether bonds); it does not refer to doubly bonded oxygen, as would be found in carbonyl groups. Alkoxy or alkoxyl is a subset of oxaalkyl that refers to groups of from 1 to 8 carbon atoms of a straight or branched configuration attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

Pharmaceutically acceptable carrier: Pharmaceutically acceptable carriers (vehicles) useful in this disclosure are known. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, molecules, or agents.

In general, the nature of the carrier will depend on the particular mode of administration being employed. For instance, injectable formulations usually include fluids that include pharmaceutically and physiologically acceptable fluids such as water, physiological saline, balanced salt solutions, aqueous dextrose, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically acceptable salt: Refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teolatic, p-toluenesulfonic, and the like. When the compounds contain an acidic side chain, for example —COOH, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′, dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Poloxamers: Nonionic triblock copolymers composed of a central hydrophobic chain of polyoxypropylene (poly(propylene oxide)) flanked by two hydrophilic chains of polyoxyethylene (poly(ethylene oxide)).

Retina: The light (photon) sensitive portion of the eye, that contains the photoreceptors (cones and rods) for light. Rods and cones perform light perception through the use of light sensitive pigments. The light sensitive pigments are made of protein called Opsin and a chromophore called retinene, which the variant is of vitamin A. The rods contain Rhodopsin while the cones contain iodopsin. Rods and cones transmit signals through successive neurons that trigger a neural discharge in the output cells of the retina and the ganglion cells. The visual signals are conveyed by the optic nerve to the lateral geniculate bodies from where the visual signal is passed to the visual cortex (occipital lobe) and registered as a visual stimulus. “Rod cells”, or “rods,” are photoreceptor cells in the retina of the eye that can function in less intense light than the other type of visual photoreceptor, cone cells. Rods are concentrated at the outer edges of the retina and are used in peripheral vision. “Cone cells,” or “cones,” are responsible for color vision and function best in relatively bright light. Cone cells are densely packed in the fovea centralis, a 0.3 mm diameter rod-free area with very thin, densely packed cones which quickly reduce in number towards the periphery of the retina. There are about six to seven million cones in a human eye and are most concentrated towards the macula. Cones are less sensitive to light than the rod cells in the retina (which support vision at low light levels) but allow the perception of color. They are also able to perceive finer detail and more rapid changes in images, because their response times to stimuli are faster than those of rods. In humans, cones are normally one of the three types, each with different pigment, namely: S-cones, M-cones and L-cones. Each cone is therefore sensitive to visible wavelengths of light that correspond to short-wavelength, medium-wavelength and long-wavelength light. The three types have peak wavelengths near 420-440 nm, 534-545 nm and 564-580 nm, respectively, depending on the individual. The Opsin or pigment is on the outer side, lying on the retinal pigment epithelium. This epithelium end contains many stacked disks containing opsins. Like cones, rod cells have a synaptic terminal, an inner segment, and an outer segment. The synaptic terminal forms a synapse with another neuron, for example a bipolar cell. The inner and outer segments are connected by a cilium. The inner segment contains organelles and the cell's nucleus, while the rod outer segment, which is pointed toward the back of the eye, contains the light-absorbing materials. Activation of photopigments by light sends a signal by hyperpolarizing the rod cell, leading to the photoreceptor cell to send less neurotransmitter to the bipolar and horizontal cells. The bipolar cell then releases its transmitter at the bipolar-ganglion synapse and excites the retinal ganglion cells which extend into the brain.

Retinal Degeneration: Deterioration of the retina, including progressive death of the photoreceptor cells of the retina or associated structures (such as retinal pigment epithelium). Retinal degeneration includes diseases or conditions such as dry age-related macular degeneration (dry AMD), Stargardt disease, Best disease, commotio retinae (trauma), myopic macular degeneration, and/or an inherited retinal dystrophy.

Retinal Pigment Epithelium (RPE): The pigmented layer of hexagonal cells (RPE cells), present in vivo, just outside of the neurosensory retina that is attached to the underlying choroid. These cells are densely packed with pigment granules, and shield the retina from excessive light. The retinal pigment epithelium also serves as the limiting transport factor that maintains the retinal environment by supplying small molecules such as amino acid, ascorbic acid and D-glucose while remaining a tight barrier to choroidal blood borne substances.

Small molecule: An organic molecule with a molecular weight of about 1000 Daltons or less.

Subject: A living multi-cellular vertebrate organism, a category that includes human, laboratory, and veterinary subjects, including human and non-human mammals.

TDI-011536: A LATS kinase inhibitor having the chemical structure of:

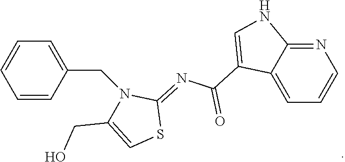 [SMILES: O=C(/N=c1\scc(CO)n1Cc1ccccc1)c1c[nH]c2ncccc12]

TDI-011536 is assigned CAS Registry No. 2687970-96-1, which is incorporated by reference herein. TDI-011536 and methods of its preparation are described in PCT Pub. No. WO2021158936 and Kastan et al., “Development of an improved inhibitor of Lats kinases to promote regeneration of mammalian organs,” Proc. Nat'l Acad. Sci. U.S.A., 119 (28) e2206113119, pages 1-7, each of which are incorporated by reference herein.

Therapeutically effective amount: An amount of a compound sufficient to treat a specified disorder or disease, or to ameliorate or eradicate one or more of its symptoms and/or to prevent the occurrence of the disease or disorder, such as retinal degeneration. The amount of a compound which constitutes a “therapeutically effective amount” will vary depending on the compound, the route of administration, the disease state and its severity, the age of the subject to be treated, and the like. The therapeutically effective amount can be determined by a person of ordinary skill in the art, for example, through various in vitro, in vivo, or ex vivo assays.

Test agent: An agent used in a test or screen, and which can be essentially any agent, such as a small molecule, a polypeptide, an antibody, a hormone, a nucleic acid, a modified nucleic acid, a sugar, a lipid and the like. Test agents are used, for example, when screening for compounds that reduce retinal degeneration in an in vitro or in vivo model of disease.

Treating, Treatment, and Therapy: Any success or indicia of success in the attenuation or amelioration of an injury, pathology, or condition, including any objective or subjective parameter such as abatement, remission, diminishing of symptoms or making the condition more tolerable to the subject, slowing in the rate of degeneration or decline, making the final point of degeneration less debilitating, improving a subject's physical or mental well-being, or improving vision. The treatment may be assessed by objective or subjective parameters; including the results of a physical examination, neurological examination, or psychiatric evaluations. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters known in the art that are specific to the particular disease, such as improved vision. A “prophylactic” treatment is a treatment administered to a subject who does not exhibit signs of a disease or exhibits only early signs for the purpose of decreasing the risk of developing pathology.

Under conditions sufficient for: A phrase that is used to describe any environment that permits the desired activity.

### II. LATS Kinase Inhibitors

LATS kinase inhibitors suitable for the present use include any compound covered by formula I:

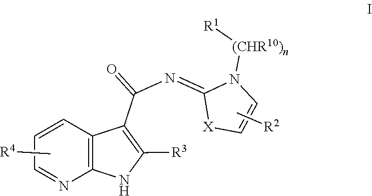

The formula I can be broken down into two subgenera. In a first subgenus, X is sulfur, and compounds are thiazol-2(3H)-ylidene)-1H-pyrrolo[2,3-b]pyridine-3-carboxamides of formula II:

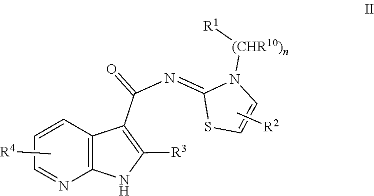

In a second subgenus, X is oxygen, and compounds are oxazol-2(3H)-ylidene)-1H-pyrrolo[2,3-b]pyridine-3-carboxamides of formula III:

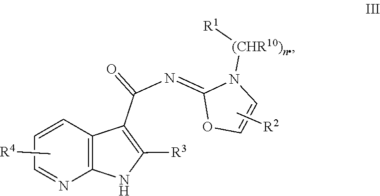

wherein n is one and R¹ is optionally substituted phenyl.

In some aspects of the formulae I and II, n may be zero. In other aspects of the formulae I-III, n may be one. In these aspects, R¹⁰ may be hydrogen.

In some aspects, R¹ is optionally substituted (C₁-C₆)alkyl, carboxy, phenyl, cyclohexyl, 5-membered heterocyclyl, 6-membered heterocyclyl or heterobicyclyl. In some non-limiting examples, R¹ may be methyl, ethyl, aminobutyl, and carboxyethyl. In other aspects, R¹ is optionally substituted cyclohexyl, or R¹ is optionally substituted phenyl, or R¹ is optionally substituted heterocyclyl, for example, pyridinyl, pyrazolyl, piperidinyl, tetrahydropyranyl, tetrahydrofuranyl, or tetrahydroisoquinolinyl. When R¹ is optionally substituted phenyl, it may carry one or two substituents selected independently from halogen, cyano, hydroxy, amino, carboxy, (C₁-C₆)hydrocarbyl, trifluoromethyl, methoxy, acetyl, formyl, hydroxy(C₁-C₃)alkyl, methoxycarbonyl [—C(═O)OCH₃], carboxamido [—C(═O)NH₂], methanesulfonylamino, and amino(C₁-C₃)alkyl. When R¹ is pyridinyl, pyrazolyl, piperidinyl, tetrahydropyranyl, or tetrahydroisoquinolinyl, each heterocycle may be optionally substituted with one or two substituents selected independently from amino, hydroxy and (C₁-C₆)hydrocarbyl.

In some aspects, R¹ is selected from carboxy and optionally substituted (C₁-C₆)alkyl, phenyl, cyclohexyl, 5-membered heterocyclyl, 6-membered heterocyclyl and heterobicyclyl. In some of these R¹ is selected from methyl, ethyl, aminobutyl, and carboxyethyl. In others R¹ is optionally substituted cyclohexyl. In some of these R¹ is optionally substituted heterocyclyl. The heterocycle may be pyridinyl, pyrazolyl, piperidinyl, tetrahydropyranyl, tetrahydrofuranyl, and tetrahydroisoquinolinyl, each optionally substituted. Optional substituents may include one or two substituents selected independently from amino, hydroxy and (C₁-C₆)hydrocarbyl.

In some aspects, R¹ is optionally substituted phenyl. The phenyl may be substituted with one or two substituents selected independently from halogen, cyano, hydroxy, amino, carboxy, (C₁-C₆)hydrocarbyl, trifluoromethyl, methoxy, acetyl, formyl, hydroxy(C₁-C₃)alkyl, methoxycarbonyl, carboxamido, methanesulfonylamino, and amino(C₁-C₃)alkyl. In some of these aspects, R¹ is phenyl substituted at the ortho position and n is zero; in others R¹ is optionally substituted phenyl and n is one.

In some aspects, R² is selected from —C(═O)O(C₁-C₆)alkyl, —C(═O)NR²⁰R²¹, and (C₁-C₆)oxaalkyl. In some of these aspects R²⁰ is selected from hydrogen and methyl, and R²¹ is selected from hydrogen, methyl, (C₁-C₆)oxaalkyl, dimethylamino(C₁-C₆)alkyl, and —C(CH₂)₂-Het. In others, R²⁰ and R²¹ taken together with the nitrogen to which they are attached form a 4-7-membered aliphatic heterocycle. Exemplary aliphatic heterocycles include piperidine, piperazine, morpholine, pyrrolidine, azetidine, azepine and the like.

In some aspects, R² is selected from hydrogen, methyl, ethyl, propyl, cyclopropyl, hydroxymethyl, and trifluoromethyl.

In some aspects, R³ and R⁴ are selected from hydrogen, chloro and methyl.

In some aspects, R¹ is selected from (C₁-C₆)alkyl, carboxy, (C₃-C₇)carbomonocyclyl, (C₉-C₁₁)carbobicyclyl, heteromonocyclyl, and heterobicyclyl, wherein the (C₁-C₆)alkyl, (C₃-C₇)carbomonocyclyl, (C₉-C₁₁)carbobicyclyl, heteromonocyclyl, and heterobicyclyl may be optionally substituted with from one to three substituents selected independently from halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, heteroaryl, benzenesulfonyl, (C₁-C₃)alkoxycarbonyl, aminocarbonyl, (C₁-C₃)alkylaminocarbonyl, di(C₁-C₃)alkylaminocarbonyl, (C₁-C₃)alkylamino, di(C₁-C₃)alkylamino, amino(C₁-C₃)alkyl, (C₁-C₃)alkylamino(C₁-C₃)alkyl, (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkylsulfonyl, phenoxy, and benzyloxy;
R² is selected from hydrogen, halogen, (C₁-C₇)hydrocarbyl, halo(C₁-C₆)alkyl, (C₁-C₅)acyl, hydroxy(C₁-C₃)alkyl, —C(═O)O(C₁-C₆)alkyl, —C(═O)NR²⁰R²¹, and (C₁-C₆)oxaalkyl;
R³ is selected from hydrogen, halogen, (C₁-C₆)hydrocarbyl, halo(C₁-C₆)alkyl, (C₁-C₆)acyl, and (C₁-C₃)alkoxy;
R⁴ is selected from hydrogen, halogen, (C₁-C₆)hydrocarbyl, halo(C₁-C₆)alkyl, (C₁-C₅)acyl, and (C₁-C₃)alkoxy;
R¹⁰ is selected independently in each instance from hydrogen and methyl;
n is zero, one or two;
m is zero, one or two; and
X is S; or, when n is 1 and R¹ is optionally substituted phenyl, X is S or O, with the proviso that, when R¹ is phenyl, X is sulfur, and n is 1, at least one of R², R³, R⁴, and R¹⁰ is other than hydrogen; and
R²⁰ is selected from hydrogen and (C₁-C₆)hydrocarbyl; and R²¹ is selected from hydrogen, (C₁-C₆)hydrocarbyl, (C₁-C₆)oxaalkyl, amino(C₁-C₆)alkyl, (C₁-C₃)alkylamino(C₁-C₆)alkyl, di(C₁-C₃)alkylamino(C₁-C₆)alkyl, and —(CH₂)ₘ-Het, wherein Het is an aliphatic mono- or bicyclic heterocycle, optionally substituted with a substituent selected from hydroxy, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, aminocarbonyl, (C₁-C₃)alkylaminocarbonyl, di(C₁-C₃)alkylaminocarbonyl, (C₁-C₃)alkylamino, and di(C₁-C₃)alkylamino; or
taken together with the nitrogen to which they are attached, R²⁰ and R²¹ form an aliphatic heterocyle.

In any of the above aspects, R² may be selected from —C(═O)O(C₁-C₆)alkyl, —C(═O)NR²⁰R²¹, and (C₁-C₆)oxaalkyl.

In any of the above aspects, R²⁰ may be selected from hydrogen and methyl, and R²¹ is selected from hydrogen, methyl, (C₁-C₆)oxaalkyl, dimethylamino(C₁-C₆)alkyl, and —(CH₂)ₘ-Het.

In any of the above aspects, R²⁰ and R²¹ may be taken together with the nitrogen to which they are attached to form a 4-7-membered aliphatic heterocycle.

In some aspects, R¹ is selected from (C₁-C₆)alkyl, carboxy, (C₃-C₇)carbomonocyclyl, (C₉-C₁₁)carbobicyclyl, heteromonocyclyl, and heterobicyclyl, wherein said (C₁-C₆)alkyl, (C₃-C₇)carbomonocyclyl, (C₉-C₁₁)carbobicyclyl, heteromonocyclyl, and heterobicyclyl may be optionally substituted with from one to three substituents selected independently from halogen, cyano, hydroxy, nitro, amino, acetoxy, carboxy, (C₁-C₇)hydrocarbyl, halo(C₁-C₆)alkyl, (C₁-C₃)alkoxy, halo(C₁-C₃)alkoxy, (C₁-C₆)acyl, (C₁-C₃)alkoxy(C₁-C₃)alkyl, hydroxy(C₁-C₃)alkyl, heteroaryl, benzenesulfonyl, (C₁-C₃)alkoxycarbonyl, aminocarbonyl, (C₁-C₃)alkylaminocarbonyl, (C₁-C₃)alkylamino, di(C₁-C₃)alkylamino, amino(C₁-C₃)alkyl, (C₁-C₃)alkylamino(C₁-C₃)alkyl, (C₁-C₃)dialkylamino(C₁-C₃)alkyl, (C₁-C₃)alkylthio, (C₁-C₃)alkylsulfonylamino, (C₁-C₃)alkylsulfinyl, (C₁-C₃)alkyl sulfonyl, phenoxy, and benzyloxy;
R² is selected from hydrogen, halogen, (C₁-C₆)hydrocarbyl, halo(C₁-C₆)alkyl, (C₁-C₆)acyl, hydroxy(C₁-C₃)alkyl, and (C₁-C₃)alkoxy;
R³ is selected from hydrogen, halogen, (C₁-C₆)hydrocarbyl, halo(C₁-C₆)alkyl, (C₁-C₆)acyl, and (C₁-C₃)alkoxy;
R⁴ is selected from hydrogen, halogen, (C₁-C₆)hydrocarbyl, halo(C₁-C₆)alkyl, (C₁-C₆)acyl, and (C₁-C₃)alkoxy;
R¹⁰ is selected independently in each instance from hydrogen and methyl;
n is zero, one or two; and
X is O or S, with the proviso that, when R¹ is phenyl, X is sulfur, and n is one, at least one of R², R³, R⁴, and R⁰ is other than hydrogen.

In any of the above aspects, n may be zero.

In any of the above aspects, n may be one.

In any of the above aspects, R¹⁰ may be hydrogen.

In any of the above aspects, R¹ may be selected from carboxy and optionally substituted (C₁-C₄)alkyl, phenyl, cyclohexyl, 5-membered heterocyclyl, 6-membered heterocyclyl and heterobicyclyl.

In any of the above aspects, R¹ may be selected from methyl, ethyl, aminobutyl, and carboxyethyl.

In any of the above aspects, R¹ may be optionally substituted cyclohexyl.

In any of the above aspects, R¹ may be optionally substituted phenyl.

In any of the above aspects, R¹ may be optionally substituted heterocyclyl.

In any of the above aspects, R¹ may be selected from pyridinyl, pyrazolyl, piperidinyl, tetrahydropyranyl, tetrahydrofuranyl, and tetrahydroisoquinolinyl, each optionally substituted.

In any of the above aspects, R¹ may be phenyl or phenyl substituted with one or two substituents selected independently from halogen, cyano, hydroxy, amino, carboxy, (C₁-C₆)hydrocarbyl, trifluoromethyl, methoxy, acetyl, formyl, hydroxy(C₁-C₃)alkyl, methoxycarbonyl, carboxamido, methanesulfonylamino, and amino(C₁-C₃)alkyl.

In any of the above aspects, R¹ may be phenyl substituted at the ortho position and n is zero.

In any of the above aspects, R¹ may be selected from pyridinyl, pyrazolyl, piperidinyl, tetrahydropyranyl, and tetrahydroisoquinolinyl, each optionally substituted with one or two substituents selected independently from amino, hydroxy and (C₁-C₆)hydrocarbyl.

In any of the above aspects, X may be S.

In any of the above aspects, X may be O.

In any of the above aspects, R³ and R⁴ may be selected independently from hydrogen, chloro and methyl.

In any of the above aspects, R² may be selected from hydrogen, methyl, ethyl, propyl, cyclopropyl, hydroxymethyl, and trifluoromethyl.

In any of the above aspects, a compound according to formula I may be selected from examples T01-T80 as provided in PCT Pub. No. WO2021158936, which is incorporated by reference herein in its entirety.

The compounds provided herein can be prepared using any suitable means, for example, as illustrated in General Schemes MV and in greater details in Schemes 1-69 described in PCT Pub. No. WO2021158936, which is incorporated by reference herein in its entirety. Detailed description for the synthesis of the intermediates and exemplified compounds are also disclosed in PCT Pub. No. WO2021158936.

### III. LATS Kinase Inhibitors for Treatment of Retinal Degeneration

Provided herein is a method of treating retinal degeneration, comprising administering a therapeutically effective amount of a LATS kinase inhibitor to a subject with retinal degeneration.

Any suitable LATS kinase inhibitor can be used in the disclosed method, such as those described in PCT Pub. No. WO2021158936. In some implementations, the LATS kinase inhibitor is TDI-011536.

In some implementations, as subject is selected for treatment using the methods provided herein, for example a subject with retinal degeneration is selected for treatment.

The disclosed methods have utility for treatment of retinal degeneration that is not due to proliferation of retinal pigment epithelium (RPE) cells. For example, in some implementations, the type of retinal degeneration treated using the disclosed method is dry age-related macular degeneration (dry AMD), Stargardt disease, Best disease, myopic macular degeneration, commotio retinae (trauma), and/or an inherited retinal dystrophy. In some implementations, the type of retinal degeneration treated using the disclosed method is early, intermediate, or late stage dry age-related macular degeneration (dry AMD).

The retinal degeneration in the subject does not need to be completely inhibited or repaired for the method to be effective. For example, the method can reduce the retinal degeneration at least 10%, at least 20%, at least 50%, at least 60%, at least 70%, at least 80%, at least 90%, at least 95%, at least 98%, or even at least 100% (elimination of retinal degeneration), as compared to the level of retinal degeneration in the absence of the treatment (e.g., in control subjects). In some aspects, the method results in an improvement in vision in the subject.

In some aspects, the method delays progression of retinal degeneration in the subject, for example by at least a month, six months, a year, two years, or more, as compared to the level of retinal degeneration in the absence of the treatment (e.g., in control subjects).

In some aspects, the subject has dry AMD and treating the retinal degeneration delays progression of dry AMD in the subject from early to intermediate stage dry AMD (such as by at least one month, at least six months, at least one year, at least two years, or longer) compared to a control.

In some aspects, the subject has dry AMD and treating the retinal degeneration delays progression of dry AMD in the subject from early to late stage dry AMD (such as by at least one month, at least six months, at least one year, at least two years, or longer) compared to a control.

In some aspects, the subject has dry AMD and treating the retinal degeneration delays progression of dry AMD in the subject from intermediate to late stage dry AMD (such as by at least one month, at least six months, at least one year, at least two years, or longer) compared to a control.

In some aspects, the subject has dry AMD and treating the retinal degeneration reduces the number and/or size of drusen (e.g., by at least 10%, at least 20%, at least 50%, at least 60%, at least 70%, at least 80%, at least 90%, at least 95%, at least 98%, or even at least 100% (elimination of drusen) under the retina in the subject compared to a control.

Efficacy of treatment can be evaluated by methods known to one of ordinary skill in the art, including, but not limited to use of fluorescent dye (such as fluorescein) for evaluating retinal cell death, optical coherence tomography (OCT) for evaluating retinal morphology, and/or electroretinogram (ERG) for evaluating retinal function. Other methods such as histology and immunofluorescent labeling of cell death markers can also be used.

Any suitable mode of administration may be used. In some implementations, the LATS kinase inhibitor is administered by intravitreal injection to the eye. In some implementations, the LATS kinase inhibitor is administered topically to the eye. In some implementation, the LATS kinase inhibitor is administered through topical route, subconjunctival route, subretinal route, periocular route, or suprachoroidal route.

Ocular drug delivery can be achieved via multiple routes of administration, such as the topical, systemic, intravitreal, and periocular routes. Topical administration, notably eye drops, is the ocular drug delivery route that guarantees high compliance by patients due to ease of use. Nowadays, intravitreal drug injections are extensively used for various retinal diseases, and intraocular pharmacokinetics and efforts to enhance the drug efficacy have been investigated. Intravitreal administration has several advantages compared to other routes; this is because drugs can be directly delivered into the vitreous and retina, ultimately avoiding the barriers of the anterior segment. Relatively lower drug doses, reasonable bioavailability, and freely manipulated drug molecules could be strengths for efficient drug delivery while minimizing toxicity. After intravitreal injection, the drug is distributed toward the anterior and posterior segments of the eye and cleared through the aqueous, ciliary body, and retina. When other administration routes are employed, the drug agents reach the posterior segment of the eye via the suprachoroidal space, subretinal space, or trans-scleral diffusion and are eliminated by both the anterior and posterior pathways. The subconjunctival route is a minimally invasive route for ocular drug delivery to the posterior segment. Drug injection or implants in the subconjunctival space skip the conjunctival and corneal barriers and display a higher permeability via the retina/choroidal area. Suprachoroidal administration is another minimally invasive technique for drug delivery to the posterior segment of the eye. After the drug is delivered to the suprachoroidal space via this pathway, it can directly target retinal layers and the choroid due to the posterior pole fluid flow, thereby avoiding multiple ocular tissue barriers and accomplishing drug efficacy at low dose concentrations. In addition, sustained release could be achieved due to drug accumulation and distribution in the suprachoroidal area. The subretinal space is located between the RPE layer and the photoreceptors. Under a surgical microscope, drug administration can be performed with direct visualization.

Any suitable dose and dosing protocol may be used to administer the therapeutically effective amount of the LATS kinase inhibitor to the subject. In some implementations, the therapeutically effective amount of the LATS kinase inhibitor is administered periodically (e.g., monthly, every two months, every three months, every six months, or every year) for a set period of time (such as six months, or 1, 2, 3, 4, 5, 6, 7, 8, 9, 10 years) or indefinitely. In some implementations, the therapeutically effective amount of the LATS kinase inhibitor is administered in no more than five doses, such as no more than four doses, no more than three doses, no more than two doses, or a single dose to treat the retinal degeneration in the subject. In some implementations, the therapeutically effective amount of the LATS kinase inhibitor is administered in one, two, three, four, or five doses.

The amount of LATS kinase inhibitor per dose may vary based various parameters, including but not limited to the type and severity of disease, the specific subject being treated, and the formulation of the active drug. In some implementations, the therapeutically effective amount of the LATS kinase inhibitor maintains about 0.5-5 μM (e.g., about 0.6 μM, about 0.7 μM, about 0.8 μM, about 0.9 μM, about 1 μM, about 1.5 μM, about 2 μM, about 2.5 μM, about 3 μM, about 4 μM, about 5 μM, about 0.5-5 μM, about 0.5-4.5 μM, about 0.5-4 μM, about 0.5-3.5 μM, about 0.5-3 μM, about 0.5-2.5 μM, about 0.5-2 μM, about 0.5-1.5 μM, about 0.5-1 μM, about 1-5 μM, about 1.5-5 μM, about 2-5 μM, about 2.5-5 μM, about 3-5 μM, or about 3.5-5 μM) of the LATS kinase inhibitor in vitreous humor for about 3-5 days (e.g., about 3, about 3.5, about 4, about 4.5, or about 5 days).

In some examples, the composition is administered intravitreally to one or both eyes of the subject. In some examples, the amount of the composition administered is about 1 μl to about 100 μl (such as about 5-50 μl, about 5-20 μl, about 1-20 μl, about 10-20 μl, or about 20-50 μl), for example, about 15 μl. In some examples, the amount of the composition administered to the subject is about 50 μl. The composition comprises an effective amount of the LATS kinase inhibitor to reduce retinal degeneration in the subject across the prescribed number of doses.

In some implementations, a dose of 1-500 μg of the LATS kinase inhibitor is administered by single or repeated intravitreal injection to the eye of the subject. In some implementations, the composition administered to the subject comprises about 1 to about 100 mM of the LATS kinase inhibitor, such as about 5 to about 50 mM LATS kinas inhibitor.

In some implementations, the LATS kinase inhibitor is administered by intravitreal injection to the eye at a dose of about 10-300 μl of about 1-50 mM TDI-011536. In some implementations, the LATS kinase inhibitor is administered by intravitreal injection to the eye at a dose of 50 μl of 15 mM TDI-011536. In some implementations, the LATS kinase inhibitor is administered by intravitreal injection to the eye at a dose of 50 μl of 45 mM TDI-011536.

In some implementations, the LATS kinase inhibitor is administered by intravitreal injection to the eye at a dose of about 0.1-2.25 μmol, e.g., about 0.1 μmol, about 0.2 μmol, about 0.3 μmol, about 0.4 μmol, about 0.5 μmol, about 0.6 μmol, about 0.7 μmol, about 0.8 μmol, about 0.9 μmol, about 1 μmol, about 1.1 μmol, about 1.2 μmol, about 1.3 μmol, about 1.4 μmol, about 1.5 μmol, about 1.6 μmol, about 1.7 μmol, about 1.8 μmol, about 1.9 μmol, about 2.0 μmol, about 2.1 μmol, about 2.2 μmol, about 2.25 μmol, about 0.1-2.2 μmol, about 0.1-2.1 μmol, about 0.1-2 μmol, about 0.1-1.9 μmol, about 0.1-1.8 μmol, about 0.1-1.7 μmol, about 0.1-1.6 μmol, about 0.1-1.5 μmol, about 0.1-1.4 μmol, about 0.1-1.3 μmol, about 0.1-1.2 μmol, about 0.1-1.1 μmol, about 0.1-1 μmol, about 0.1-0.9 μmol, about 0.1-0.8 μmol, about 0.1-0.7 μmol, about 0.1-0.6 μmol, about 0.1-0.5 μmol, about 0.1-0.4 μmol, about 0.1-0.3 μmol, about 0.1-0.2 μmol, about 0.2-2.25 μmol, 0.3-2.25 μmol, 0.4-2.25 μmol, 0.5-2.25 μmol, 0.6-2.25 μmol, 0.7-2.25 μmol, 0.8-2.25 μmol, 0.9-2.25 μmol, 1-2.25 μmol, 1.1-2.25 μmol, 1.2-2.25 μmol, 1.3-2.25 μmol, 1.4-2.25 μmol, 1.5-2.25 μmol, 1.6-2.25 μmol, 1.7-2.25 μmol, 1.8-2.25 μmol, 1.9-2.25 μmol, 2-2.25 μmol, or 2.1-2.25 μmol.

In some implementations, the LATS kinase inhibitor is administered in an amount sufficient to maintain about 0.5 to about 5 μM (such as about 0.5 μM, about 1 μM, about 2 μM, about 3 μM, about 4 μM, or about 5 μM) of the LATS kinase inhibitor in the neuroretina of the subject for about 3 to about 15 (such as about 3, about 4, about 5, about 6, about 7, about 8, about 9, about 10, about 11, about 12, about 13, about 14, or about 15) days following administration.

In some implementations, the LATS kinase inhibitor is administered in an amount sufficient to induce a short-term increase (such as up to 30 days, up to 20 days up to 15 days, up to 10 days, or up to 5 days) in proliferation of retinal pigment epithelium (RPE) cells and/or Müller glia cells in sites of retinal degeneration.

The LATS kinase inhibitor can be formulated in any suitable pharmaceutical composition for use in the disclosed methods.

The pharmaceutical compositions may be formulated in a variety of ways depending, for example, on the mode of administration (e.g., by intravitreal injection). Parenteral formulations may comprise injectable fluids that are pharmaceutically and physiologically acceptable fluid vehicles such as water, physiological saline, other balanced salt solutions, aqueous dextrose, glycerol or the like. Excipients may include, for example, nonionic solubilizers, or proteins, such as human serum albumin or plasma preparations. If desired, the pharmaceutical composition to be administered may also contain non-toxic auxiliary substances, such as wetting or emulsifying agents, preservatives, and pH buffering agents and the like, for example, sodium acetate or sorbitan monolaurate.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. For parenteral administration, bolus injection or continuous infusion may be used. The LATS kinase inhibitor may be in powder form for reconstitution with a suitable vehicle, e.g. sterile water, before use.

In some implementations the LATS kinase inhibitor is administered as an aqueous suspension formulation prepared with poloxamer solubilizing reagents (P 188 or similar). Poloxamers are biocompatible, and frequently included in ophthalmic formulations to improve the ocular bioavailability of drugs by increasing vehicle viscosity. Poloxamers are chemically synthesized, nonionic, triblock (ABA type) in nature made out of ethylene oxide (E)) and propylene oxide (PO) unit organized in EOₓ-PO_{y}-EOₓ sequence. Their chemical formula is 110 [CH₂—CH₂O]ₓ[CH(CH₃)—CH₂O]_{y}[CH₂—CH₂O]ₓOH, where y is higher than 14. These copolymers are commercially accessible under trademarks of Pluronic, Synperoni, Tetronic, Kolliphor, etc. in the liquids (L) pastes (P) and flakes (F) form. Poloxamer 407 (P 407) and poloxamer 188 (P 188) are among the most commonly used poloxamers in ocular drug delivery. Poloxamer 188 (P 188) is an FDA-approved poloxamer having an average molecular weight of 8400 Daltons. Due to its amphiphilic nature and high Hydrophile-Lipophile Balance (HLB) value of 29, P188 is used as a stabilizer/emulsifier in many cosmetics and pharmaceutical preparations. P407, having an average molecular weight of 12,600 Da, is highly soluble in water and forms a transparent gel, so does not hamper normal vision. Strength and viscosity of P407 gels improves with increasing total poloxamer content in the solution. Poloxamer 407 is approved by the FDA for use as an excipient in a range of pharmaceutical dosage forms, and is listed in the Inactive Ingredient Database (TID).

In some implementations, the formulation comprises one or more pharmaceutically acceptable excipients, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opacifying agents, glidants, processing aids, colorants, sweeteners, taste-masking agents, perfuming agents, flavoring agents, polishing agents, polymer matrix systems, plasticizers and other known additives.

In some implementations, the LATS kinase inhibitor is formulated together with any suitable ocular drug delivery system, to enhance intraocular pharmacokinetics and pharmacodynamics. Such systems include but are not limited to sustained-release intravitreal implants, micro- and nanoparticles (including dendrimer, liposome, polymeric micelles, polymeric nanoparticles, solid lipid nanoparticles, coated nanoparticles, inorganic nanoparticles), and hydrogels. Microparticles and nanoparticles have been engineered to deliver drugs efficiently into the intraocular space and can encapsulate different types of molecules. Owing to the modification and fabrication of particle surfaces, compositions, polysaccharide mixtures, and ionic charges, these injected particles are distributed in the vitreous, enabling prolonged intravitreal half-lives by sustained drug release and delayed degradation and elimination.

The amount of LATS kinase inhibitor administered will depend, for example, on the subject being treated, the target (e.g., eye affected or at risk of retinal degeneration), patient condition, and the manner of administration. Within these bounds, the formulation to be administered will contain a quantity of the LATS kinase inhibitor in an amount effective to provide a therapeutically effective dose of the drug to the subject being treated.

In some examples, the subject receives the treatment unless or until a therapeutic effect is no longer observed.

The present application also provides LATS kinase inhibitors, or compositions comprising a LATS kinase inhibitor for use in treating retinal degeneration, as well as uses of a LATS kinase inhibitor in manufacture of a medicament for treating retinal degeneration. The LATS kinase inhibitors, the retinal degeneration, the applicable composition formulations, etc. are described above.

### IV. Methods to Identify an Agent for Treating Retinal Degeneration

TDI-011536 is shown in the examples to be effective for treating retinal degeneration in both in vitro and in vivo models of disease. Based on these findings, also provided herein are screening methods to identify additional agents useful to (or with the potential to) treat retinal degeneration, using TDI-011536 as a reference standard.

In some aspects, an in vitro method of identifying an agent for treating retinal degeneration is provided based on the “scratch” protocol described in the Examples. The method comprises providing a monolayer of retinal pigment epithelium (RPE) cells (such as primary human fetal RPE cells) grown in tissue culture. The RPE cells are grown under conditions sufficient for monolayer formation. The monolayer is a layer of cells that is generally one cell deep and that occupies substantially all of a target surface on which the cells are grown. The monolayer of RPE cells is scraped to produce a scraped RPE cell monolayer containing an RPE cell-free area within the monolayer. The RPE cell-free area is surrounded by RPE cells that were not removed from the monolayer during the scraping process. The scraped RPE cell monolayer is then incubated in the presence of a test agent (such as a small molecule LATS kinase inhibitor). In some aspects, this incubation step is performed in serum-free media to limit the proliferative effects of the tissue media and facilitate detection of RPE cell proliferation triggered by the test agent. The incubation step may be performed for any suitable amount of time that allows for growth and proliferation of the RPE cells (e.g., from 1 to 10 days, such as about 1, about 2, about 3, about 4, about 5, about 6, about 7, about 8, about 9, or about 10 days). Any such proliferation is detected and measured to determine if the test agent has any effect on the proliferation of the RPE cells. Proliferation can be monitored over time, such as every 12 hours, every day, or every other day.

Any suitable assay may be used to detect proliferating cells in the in vitro methods provided herein. In an example, cell proliferation is quantified as the percentage of 5-ethynyl-2′-deoxyuridine positive (EdU+) cells within a target region (such as the scratched area with RPE cells removed) of the RPE monolayer used in the in vitro method. 5-Ethynyl-2′-deoxyuridine is a thymidine analogue that is incorporated into the DNA of dividing cells. Thus, its detection identifies dividing/proliferating cells. In another approach, cell proliferation may be measured by detecting the “filling-in” of the scratched are of the monolayer area using microscopy.

Next, the measured proliferation of RPE cells in the RPE cell-free area in the presence of the test agent is compared with a control. The control represents proliferation of RPE cells in the RPE cell-free area when the scraped RPE cell monolayer is incubated in the presence of the LATS kinase inhibitor TDI-011536 (e.g., at a similar concentration as the test agent, such as concentration of 1 μM). If proliferation of RPE cells in the RPE cell-free area in the presence of the test agent is greater than proliferation of RPE cells in the RPE cell-free area in the presence of TDI-011536, then the test agent is identified as an agent for treating retinal degeneration. If proliferation of RPE cells in the RPE cell-free area in the presence of the test agent is not greater than proliferation of RPE cells in the RPE cell-free area in the presence of TDI-011536, then the test agent is not identified as an agent for treating retinal degeneration.

In some aspects, the in vitro method of identifying an agent for treating retinal degeneration further comprises comparing the proliferation of RPE cells outside of the RPE cell-free area (for example, in a portion of the RPE monolayer not affected by the scraping step) with a control, wherein the control represents proliferation of RPE cells outside of the RPE cell-free area when the scraped RPE cell monolayer is incubated in the presence of the LATS kinase inhibitor TDI-011536 (e.g., at a similar concentration as the test agent, such as concentration of 1 μM). As described in the examples, TDI-011536 treatment does not lead to over-proliferation of RPE cells in areas of the RPE monolayer unaffected by the scratch. Thus, in some aspects, the method also requires that the test agent leads to no more proliferation of RPE cells outside of the RPE cell-free area than that observed in the presence of TDI-011536.

In some aspects, an in vivo method of identifying an agent for treating retinal degeneration is provided based on the “laser photocoagulation” protocol described in the Examples. The method comprises providing an animal model of retinal degeneration in a human, wherein an eye of the animal has an area of damaged retinal pigment epithelium. Any suitable animal model may be used, such as a rabbit model. The damage to the retina in the animal model typically is acute damage, such as acute retinal damage due to laser photocoagulation as described in the Examples. A test agent (such as a small molecule LATS kinase inhibitor) is administered to the eye of the animal model that contains the damaged retinal pigment epithelium. The test agent may be administered using any suitable approach that delivers the agent to the damaged retinal pigment epithelium, for example, by intravitreal injection to the affected eye.

Next, any proliferation of RPE cells in the damaged area of the retinal pigment epithelium is detected and measured as an indication of wound healing. Any suitable assay may be used to detect proliferating RPE cells in the in vivo methods. In an example, RPE cell proliferation is assessed using retinal histology and immunohistochemistry. In another example, OCT imaging is used to examine the damaged retina and observe any healing as an indication of RPE proliferation. The measured proliferation of RPE cells in the damaged area of the retinal pigment epithelium in animals treated with the test agent is compared with a control. The control represents proliferation of RPE cells in the damaged area of the retinal pigment epithelium in animals treated with the LATS kinase inhibitor TDI-011536 (e.g., at a similar dose as the test agent, such as a dose of 50 μL of 15 mM TDI-011536). If proliferation of RPE cells in the damaged area of the retinal pigment epithelium in animals treated with the test agent is greater than proliferation of RPE cells in the damaged area of the retinal pigment epithelium area in animals treated with TDI-011536, then the test agent is identified as an agent for treating retinal degeneration. If proliferation of RPE cells in the damaged area of the retinal pigment epithelium in animals treated with the test agent is not greater than proliferation of RPE cells in the damaged area of the retinal pigment epithelium area in animals treated with TDI-011536, then the test agent is not identified as an agent for treating retinal degeneration.

In an alternate approach, proliferation of Müller glia cells in the damaged area of the retinal pigment epithelium is detected and measured as an indication of wound healing. Any suitable assay may be used to detect proliferating Müller glia cells in the in vivo methods. In an example, Müller glia cell proliferation is assessed using retinal histology and immunohistochemistry. In another example, OCT imaging is used to examine the damaged retina and observe any healing as an indication of Müller glia proliferation. The measured proliferation of Müller glia cells in the damaged area of the retinal pigment epithelium in animals treated with the test agent is compared with a control. The control represents proliferation of Müller glia cells in the damaged area of the retinal pigment epithelium in animals treated with the LATS kinase inhibitor TDI-011536 (e.g., at a similar dose as the test agent, such as a dose of 50 μL of 15 mM TDI-011536). If proliferation of Müller glia cells in the damaged area of the retinal pigment epithelium in animals treated with the test agent is greater than proliferation of Müller glia cells in the damaged area of the retinal pigment epithelium area in animals treated with TDI-011536, then the test agent is identified as an agent for treating retinal degeneration. If proliferation of Müller glia cells in the damaged area of the retinal pigment epithelium in animals treated with the test agent is not greater than proliferation of Müller glia cells in the damaged area of the retinal pigment epithelium area in animals treated with TDI-011536, then the test agent is not identified as an agent for treating retinal degeneration.

The test agents which may be screened in accordance with this disclosure include, but are not limited to, small molecules, polypeptides, hormone, nucleic acid, modified nucleic acids, sugars, and lipids. In some aspects, the test agent is a small molecule LATS kinase inhibitor. In some aspects, the test agent is an antibody, including, but not limited to, polyclonal, monoclonal, humanized, anti-idiotypic, chimeric or single chain antibodies, and Fab, F(ab′)₂ and Fab expression library fragments, and epitope-binding fragments thereof.

Test agents identified as useful to treat retinal degeneration using the provided methods may be selected for additional study, for example, for further assessment in animal models, or for clinical assessment in humans.

### EXAMPLES

The following examples are provided to illustrate particular features of certain aspects of the disclosure, but the scope of the claims should not be limited to those features exemplified.

An in vitro wound healing assay in the retinal pigment epithelium (RPE) cultures was performed using the WoundMaker tool (Sartortius; Bohemia, NY). Wound closure was quantified as the change in wound area over time, expressed as a percentage. RPE proliferation was quantified as the percentage of 5-ethynyl-2′-deoxyuridine positive (EdU+) cells. Acute retinal injury in vivo was induced in Dutch belted rabbits via laser photocoagulation using an Iridex green laser indirect ophthalmoscope. Following the procedure, eyes received either intravitreal LKI or vehicle. Fundus photos and optical coherence tomography (OCT) imaging were obtained following the laser as well as one and four weeks later. Immunohistological analysis of frozen eye cross-sections was performed to assess RPE proliferation as determined by the cross-sectional area and the percentage of Ki67+ cells.

In vitro RPE monolayers showed increased wound healing with LKI (n=28 regions) compared to control (n=30 regions) at 24 hours (25.06±0.96 SE v. 16.81±1.12; P<0.0001), 48 hours (58.72±1.37 v. 41.19±1.47; P<0.0001), 72 hours (85.14±1.16 v. 65.36±1.45; P<0.0001), and 96 hours (91.14±1.17 v. 77.12±1.49; P<0.0001). RPE monolayers show increased cell proliferation with LKI (n=27 regions) compared to control (n=27 regions) in scratch regions (40.11±2.07 v. 33.23±1.86; P=0.04) and proximal regions (10.25±1.30 v. 2.89±0.41; P<0.0001) but not distal regions (3.20±0.48 v. 2.29±0.34; P=0.14). One week after laser injury in an in vivo model of retinal degeneration, eyes treated with LKI (n=4 eyes) demonstrated hyperpigmentation of laser spots on fundus photos compared to control (n=8 eyes). On OCT, eyes treated with LKI showed thickening of the RPE layer with associated shadowing compared to control where hypertransmission defects are apparent. After four weeks, these differences became more pronounced with LKI (n=6 eyes) compared to control (n=9 eyes). Immunohistological data corroborated these findings. After 1 week, laser spots in eyes injected with LKI (n=15 laser spots from 4 eyes) demonstrated an increase in underlying RPE cross-sectional area (P<0.0001) and percentage of Ki67+ cells within the RPE layer (P<0.001) compared to control (n=9 laser spots from 4 eyes). Additionally, Sox2+ and Ki67+Müller glia were observed in the LKI-injected eyes, but not in the controls. After 4 weeks, laser spots in eyes injected with LKI (n=10 laser spots from 5 eyes) continued to show an increase in RPE cross-sectional area (P<0.001) compared to control (n=9 laser spots from 4 eyes). However, there was no significant difference in the percentage of Ki67+ cells within the RPE layer (P=0.36) or in Müller glia. In undamaged regions, there were no Ki67+ cells within the RPE layer or Müller glia after 1 week or 4 weeks.

Hippo pathway inhibition via LKI induces increased wound healing and RPE proliferation in RPE monolayers in vitro. In addition, intravitreal LKI induces changes consistent with RPE regeneration as well as Müller glia proliferation in an in vivo model of retinal degeneration.

### Example 1

### Materials and Methods

The following example provides a brief description of the materials and methods used for the in vitro retinal scratch assay and the in vivo retinal injury assay described herein.

### RPE Cell Culture

Primary human fetal RPE were cultured in a 5% CO₂ incubator at 37° C. Cells were differentiated and once differentiated, cells were cultured in media consisting of Gibco MEM Alpha, with 5% heat-inactivated fetal bovine serum (FBS), penicillin/streptomycin, non-essential amino acids, and other factors.

Additionally, induced pluripotent stem cell (iPSC) derived RPE were cultured in a 5% CO₂ incubator at 37° C. WTC11 cells were purchased from Coriell Institute (Camden, NJ, USA). Cells were differentiated and once differentiated, cells were cultured in X-VIVO media.

Both primary human fetal RPE and iPSC-derived RPE were seeded on Matrigel-coated glass-bottom 96-well plates at a density of 100,000 cells/mm². Cells were maintained in their respective culture media for 4-6 weeks until they formed a mature, confluent monolayer. All stem cell cultures used in this study underwent 3 to 5 passages. A list of culture materials and reagents are provided in Table 1.

TABLE 1

| Name of | | Catalog | |
|---|---|---|---|
| Material/Equipment | Company | Number | Note |
| Phosphate buffered | Corning | 21-030-CV | Used in preparation of fixative and |
| saline, 1x | | | cryoprotectant |
| Paraformaldehyde | Electron | 15713 | Used as tissue fixative (diluted to |
| 20% solution | Microscopy | | 4% in PBS) |
| | Sciences | | |
| Kolliphor P188 | BASF Pharma | 30631540 | Vehicle for LKI |
| Triton X-100 | Millopore | 9036-19-5 | Used in blocking buffer |
| | Sigma | | |
| Bovine serum | VWR | 0332-100G | Used in blocking buffer |
| albumin | | | |
| Glass bottom 96 well | Porvair | 324001 | For RPE cell culture |
| microplate | Sciences | | |
| MEM alpha | Gibco | 12571064 | Used in primary human fetal RPE |
| | | | cell culture |
| N2 | Life | 17502048 | Used in primary human fetal RPE |
| | Technologies | | cell culture |
| Non-essential amino | Life | 11140050 | Used in primary human fetal RPE |
| acids | Technologies | | cell culture |
| Sodium pyruvate | Thermofisher | 11360-070 | Used in primary human fetal RPE |
| | | | cell culture |
| Penicillin- | Life | 15140122 | Used in primary human fetal RPE |
| streptomycin | Technologies | | cell culture |
| Taurine | Sigma | T-0625 | Used in primary human fetal RPE |
| | Millipore | | cell culture |
| Hydrocortisone | Sigma | H6909 | Used in primary human fetal RPE |
| | Millipore | | cell culture |
| T3 | Sigma | T5516 | Used in primary human fetal RPE |
| | Millipore | | cell culture |
| X-VIVO cell media | Lonza | 02-060Q | For iPSC derived RPE cell culture |
| Fetal bovine serum | Gibco | 26140079 | For primary human fetal RPE and |
| | | | iPSC derived cell culture |
| WoundMaker Tool | Essen | 4563 | For wound healing assay |
| | Bioscience | | |
| 1 cc syringe | Exel | 22-841-001 | For intravitreal injection |
| | International | | |
| 100 mm culture dish | Corning | 353025 | Used for rabbit eye dissection |
| Scalpel blade no. 15 | Feather | 08-916-5D | Used for rabbit eye dissection |
| Dissection | Zeiss | Stemi 2000-C | Used for rabbit eye dissection |
| microscope | | | |
| Forceps | Fine Science | 91150-20 | Used for rabbit eye dissection |
| | Tools | | |
| OmniPur Sucrose | Millipore | 167117 | Used for cryoprotectant |
| Cryostat | Leica | CM1850 | For cryosectioning |
| Superfrost Plus | Fisher | 12-550-15 | For cryosectioning |
| Microscope Slides | Scientific | | |
| High-profile | Leica | 14035838926 | For cryosectioning |
| disposable cryostat | Microsystems | | |
| blades | Inc. | | |
| Peel-A-Away | Polysciences, | 18646A | Used as embedding mold |
| Disposable | Inc. | | |
| Embedding Mold | | | |
| (22 × 22 × 20 mm Deep) | | | |
| Tissue-Tek O.C.T. | Sakura | 4583 | Used as embedding media |
| Compound | | | |
| DAPI | Fisher | D3571 | Diluted 1:300 in PBS for RPE |
| | Scientific | | monolayers, 1:1,000 in blocking |
| | | | buffer for rabbit cryosections |
| EdU Alexa Fluor 555 | Invitrogen | C10338 | Used for RPE monolayer |
| Imaging Kit | | | proliferation assay |
| Rabbit anti-Pax-6 | BioLegend | 901301 | Diluted 1:300 in blocking buffer |
| Rat anti-Ki-67 | eBioscience | 14-5698-82 | Diluted 1:100 in blocking buffer |
| Goat anti-Sox2 | R&D Systems | AF2018 | Diluted 1:100 in blocking buffer |
| Mouse anti-RPE65 | Novus Bio | NB100-355SS | Diluted 1:100 in blocking buffer |
| Donkey anti-Goat | Fisher | A-11055 | Diluted 1:1,000 in blocking buffer |
| 488 | Scientific | | |
| Donkey anti-Mouse | Fisher | A-31570 | Diluted 1:1,000 in blocking buffer |
| 555 | Scientific | | |
| Donkey anti-Rabbit | Fisher | A21206 | Diluted 1:1,000 in blocking buffer |
| 488 | Scientific | | |
| Donkey anti-Rat 647 | Fisher | A-48265 | Diluted 1:1,000 in blocking buffer |
| | Scientific | | |

### In Vitro Wound Healing Assay

Approximately 72 hours before wound induction, culture media for primary human fetal RPE either remained supplemented with 5% FBS as above (FBS-containing condition) or was replaced with culture media not containing FBS (FBS-free condition). Culture media for iPSC-derived RPE either remained as X-VIVO media as above (FBS-free condition) or was supplemented with 5% FBS (FBS-containing condition).

Following 72-hour acclimation period, RPE monolayers were then subjected to a controlled horizontal scratch using the WoundMaker Tool™ (Essen BioScience Ltd., Welwyn Garden City, United Kingdom) according to the protocol provided by the manufacturer. Wells were subsequently washed 1-2 times with their respective media in order to remove cell debris. Cells were then treated with either 1 μM DMSO (as control) or 1 μM TDI-011536 dissolved in either FBS-containing or FBS-free media. To label and track proliferating cells, cells were incubated with 10 μM 5-Ethynyl-2′-deoxyuridine (EdU) for 96 hours.

Each well was imaged immediately following the scratch injury with a DMI6000B microscope equipped with 10×/0.3 HC PL FLUOTAR Ph1 (Leica Microsystems, Buffalo Grove, IL) and ORCA-Flash4.0 LT camera (Hamamatsu Corp., Bridgewater, NJ). Along the length of each scratch, 2-3 ROIs measuring 1100 μm in length were imaged again 24, 48, 72, and 96 hours after the scratch injury. All ROIs were positioned in the central portion of the scratch. Coordinates of ROIs within each well were stored using Leica Application Suite X software. Non-uniform scratches and/or those with small width or resulting in monolayer detachment were excluded.

### Quantification of In Vitro Wound Healing

Scratch areas in μm² for each ROI were obtained using the Wand Tool in QuPath at each time point (e.g., 0, 24, 48, 72, and 96 hours after the scratch). Wound healing was quantified as the difference in scratch area at each time point normalized to (e.g., divided by) the total area immediately post-scratch, expressed as a percentage.

### In Vitro Immunofluorescence Staining

Ninety-six hours after the scratch, cells were immediately fixed with 4% paraformaldehyde (PFA) in phosphate-buffered saline (PBS) for 15 minutes at room temperature (RT). Cells were washed three times with PBS in between each step. Following fixation, cells were blocked and permeabilized with 3% bovine serum albumin (BSA) and 0.1% Triton X-100 in PBS for 2 hours at RT. Cells were then incubated with an EdU reaction cocktail according to the manufacturer's instructions for 30 minutes at RT, protected from light. Then, cells were incubated overnight with primary antibodies diluted in 3% BSA in PBS. The following morning, cells were incubated with secondary antibodies diluted in 3% BSA in PBS for 2 hours at RT, protected from light. Cells were counterstained with DAPI and then washed. A list of reagents and dilutions used can be found in Table 1.

### In Vitro Brightfield and Fluorescence Microscopy

Fluorescence images were acquired with a DMI6000B microscope equipped with 10×/0.3 HC PL FLUOTAR Ph1 lenses (Leica Microsystems, Buffalo Grove, IL) and ORCA-Flash4.0 LT camera (Hamamatsu Corp., Bridgewater, NJ). Fluorescence excitation and emission bands were as follows: 360/40 and 470/40 nm for DAPI; 470/40 and 525/50 nm for Alexa Fluor 488; 545/40 and 610/75 nm for Cy3. The system was controlled with LAS X 3.6 software. Fluorescence images were obtained using appropriate filters. In each replicate experiment, identical lighting and digital settings were kept for all experimental conditions.

### Quantification of In Vitro Cell Proliferation and PAX6 Expression

Regions measuring 800 μm² at various distances from the scratch within each well were analyzed. In uninjured wells, three regions were randomly selected within each well. In scratched wells, three regions were centered within the original scratch area (scratch region), which was determined by overlaying the brightfield image taken immediately post-scratch over the fluorescence images 96 hours post-scratch. Another three regions were selected immediately adjacent to the original scratch area but not within it (proximal region). Finally, three additional regions at least 800 μm from the original scratch area were selected (distal region). Apart from scratch regions, only regions that were intact and confluent immediately post-scratch were analyzed. Regions with significant cell loss or those that were otherwise not confluent following the scratch were excluded from analysis.

Within each region, cell proliferation was quantified by dividing EdU/DAPI doubly positive cells by the total number of DAPI+ cells, expressed as a percentage. PAX6 expression was quantified as the ratio of PAX6+ nuclei among EdU/DAPI doubly positive nuclei, also expressed as a percentage.

### In Vivo Laser Injury Model

All procedures were carried out in compliance with and approved by the Institutional Animal Care and Use Committee (IACUC) of the University of Southern California and conducted in accordance with the guidelines of the ARVO in the Statement for the Use of Animals in Ophthalmic and Visual Research.

Laser photocoagulation was used as a model for RPE injury in vivo. Prior to laser photocoagulation, Dutch-belted rabbits were sedated with ketamine and xylazine. Eyes were anesthetized with topical proparacaine and dilated with phenylephrine and tropicamide. Lubricating drops and gel were applied to prevent desiccation of the cornea.

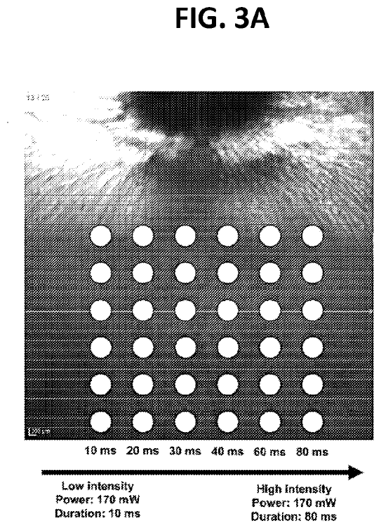

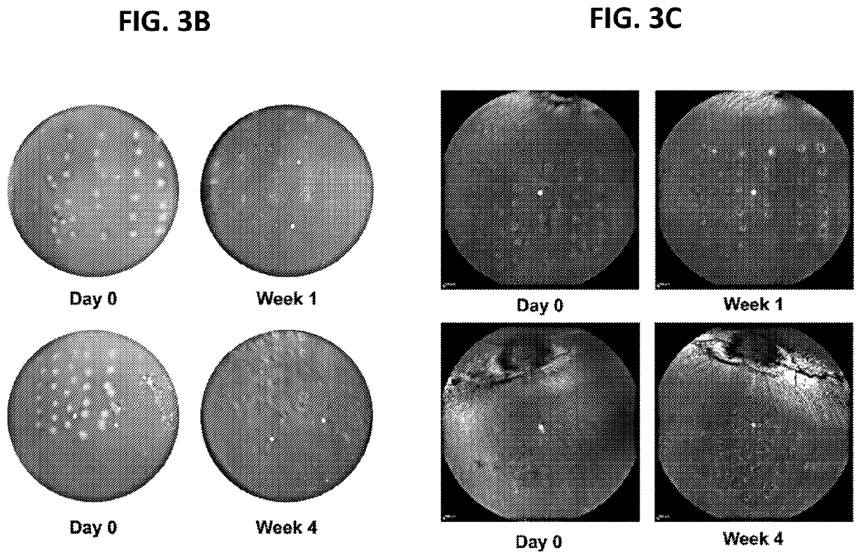

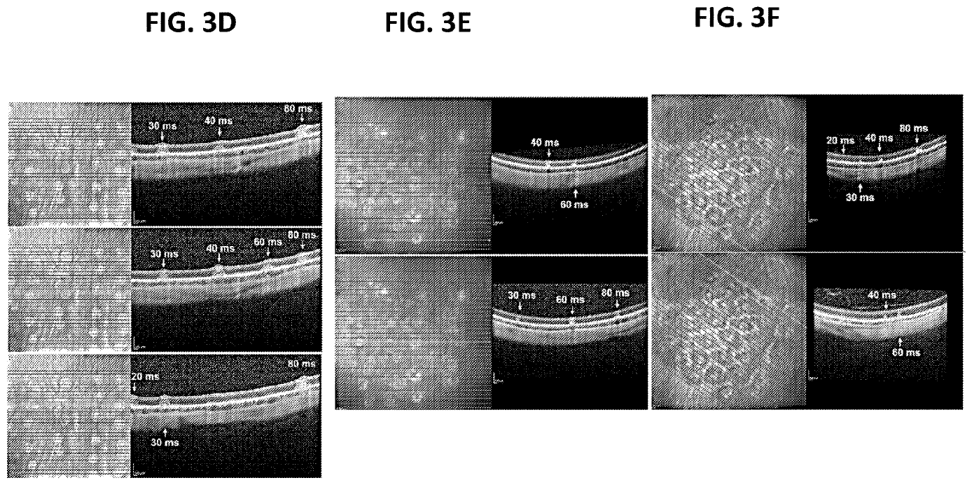

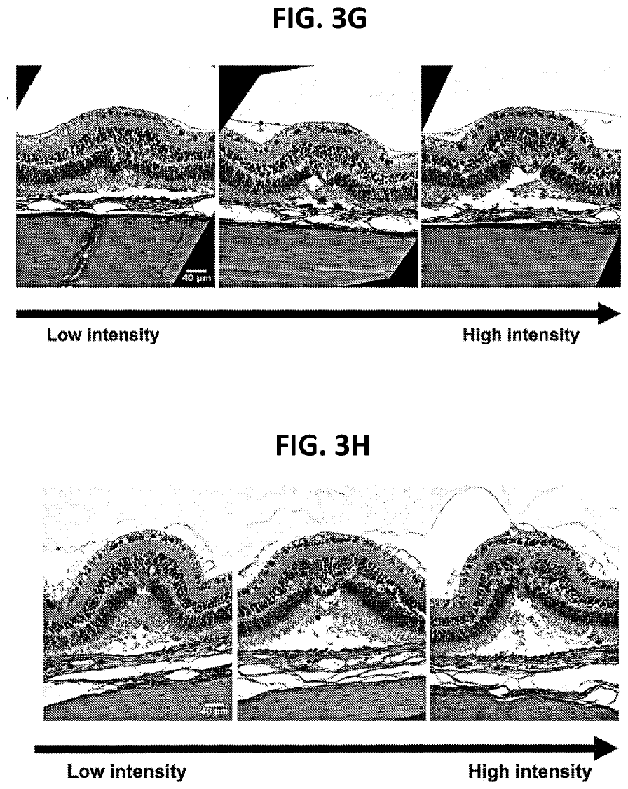

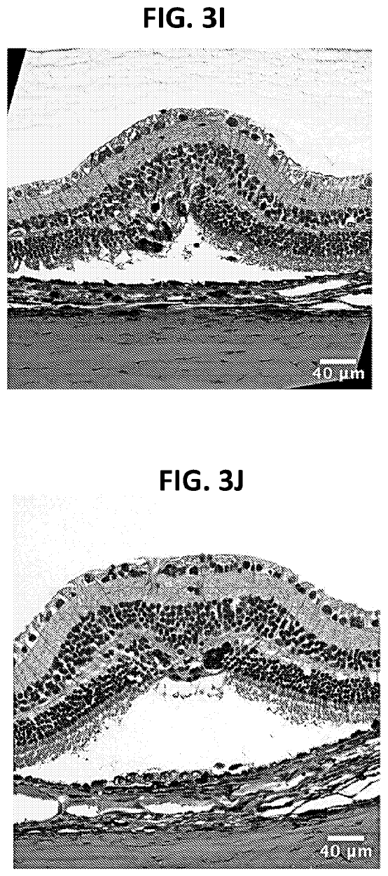

To evaluate the effects of retinal laser injury in vivo, Dutch-belted rabbits received a 6×6 grid pattern of varying spot intensities. Power was maintained constant at 170 mW while pulse duration was increased in increments of 10 or 20 ms from 10 ms in the posterior-most column to 80 ms in the anterior-most column of laser spots. Laser photocoagulation was performed with a 532 nm green laser (IRIDEX Oculight Tx) in both eyes using an indirect ophthalmoscope. Various laser intensities were titrated according to previously reported changes in retinal morphology. The effect of different laser intensities were recorded by fundus photos, OCT, as well as histological sections (FIGS. 3A-3J). Following the laser procedure, color fundus photos of both eyes were obtained using a 28 diopter lens and smartphone. OCT images of both eyes were obtained on a SPECTRALIS imaging system. Color photos and OCT images were again obtained at 1- and 4-week time points.

To evaluate the effect of LATS kinase inhibition on the RPE injury response in vivo, following sedation and pharmacologic dilation, laser photocoagulation was delivered in a 6×6 grid pattern to both eyes with a 532 nm green laser (Iridex Corporation, Mountain View, CA, USA) mounted on an indirect ophthalmoscope. Power was set to 170 mW and pulse duration was set to 40 ms. Following the laser procedure, fundus photos of both eyes were obtained using a 28-diopter lens and smartphone. OCT images of both eyes were obtained on a SPECTRALIS (Heidelberg Engineering, Heidelberg, Germany) imaging system. Fundus photos and OCT images were again obtained at 1- and 4-week time points.

### Lats Kinase Inhibitor Administration

Dutch-belted rabbits were sedated and underwent photocoagulation of both eyes as described above, with the exception of lesion intensity alteration. Instead, laser power was held at 170 mW and pulse duration remained at 40 ms.

Following the laser procedure, the eyes were anesthetized once more with topical proparacaine. Povidone-iodine was applied to the conjunctiva or sclera using sterile Q-tips. Either 50 μL of 15 mM LATS kinase inhibitor (LKI), or vehicle control, was injected per eye using 1 mL Luer-lock syringe with a 27G 1½ inch needle. The second generation LKI, TDI-011536, was used as a micro-suspension formulation prepared in phosphate buffer solution (PBS; Sigma) supplemented with 0.5% carboxymethyl cellulose (Sigma) and 0.5% Kolliphor P 188 Bio (BASF Pharma). Intravitreal injections containing LKI were administered in the right eye and control injections were administered in the left eye. Injections were administered 1-2 mm posterior to the superotemporal or superonasal limbus and delivery of each compound was confirmed on indirect ophthalmoscopy.

### In Vivo Retinal Histology and Immunohistochemistry

At 1- or 4-week time points, rabbits were euthanized with phenytoin and pentobarbital overdose. Immediately following euthanasia, eyes were enucleated using curved scissors and mobilized with forceps. Eyes used for hematoxylin and eosin (H&E) staining were immediately placed in 100 mL Davidson's fixative. Eyes used for H&E staining were fixed for 24 hours at RT. Following fixation, the cornea and lens were removed, and the eye cup was processed with a series of increasing ethanol concentrations, followed by xylene and paraffin. Tissue was sectioned at 5 μm thickness, mounted, and stained with H&E according to a routine protocol.

Eyes used for immunofluorescence staining were processed according to a previously described protocol. Briefly, eyes were immediately placed in 50 mL 4% PFA in PBS on ice. After 15 minutes, eyes were removed from 4% PFA in PBS and the cornea and iris were removed under a dissecting microscope. With the cornea and iris removed, eyes were placed back in 50 mL 4% PFA and agitated overnight at 4° C. The following morning, the lens was removed. Eyes were then placed in 50 mL of 30% sucrose in PBS at 4° C. for cryoprotection. Once cryoprotection was complete, eyes were removed from 30% sucrose solution and embedded in optimal cutting temperature (OCT) compound.

Following embedding, eyes were mounted on a Leica cryostat (Leica Microsystems, Wetzlar, Germany) for sectioning. All retinal sections were 14 μm thickness and attached to a glass slide. A list of materials used can be found in Table 1.

### In Vivo Brightfield and Fluorescence Microscopy

Confocal z-stacks were acquired with an LSM 800 system (Carl Zeiss Microscopy). Laser light of 405, 488, 555 and 647 nm was used to excite fluorescence of DAPI and Alexa Fluor 488, 555 and 647, respectively. The laser power and gain were adjusted to maximize dynamic range without saturating any pixels. After image capture, brightness adjustments, image cropping, channel overlays and maximum intensity projections were generated using FIJI ImageJ software.

### Quantification of In Vivo Cell Proliferation

To quantify RPE proliferation at the site of laser injury in vivo, regions 400 μm in width were centered on the outer nuclear layer (ONL) defect. Laser spots with ONL defects <30 μm in diameter, retinal detachments >225 μm, or an adjacent laser spot <200 μm away from the center of the ONL defect were excluded from analysis. Additionally, laser spots with significant tissue folding or tears were excluded. RPE cross-sectional area in μm² was obtained using the Wand Tool in QuPath using the RPE65 channel. RPE proliferation was quantified as the number of Ki67+ cells divided by DAPI+ cells within the RPE layer, expressed as a percentage. Choroidal proliferation was quantified as the number of Ki67+ cells divided by DAPI+ cells in the choroid.

To evaluate the effect of LKI on Müller glia at the site of injury, glial cell proliferation was quantified as the number of Ki67/Sox2 doubly positive cells divided by total number of Sox2/DAPI doubly positive cells within the 400 μm laser region, expressed as a percentage.

To evaluate the effect of LKI in undamaged retina, regions 400 μm in width in the peripheral retina were analyzed. The percentages of Ki67+ cells in the RPE layer, Ki67/Sox2 doubly positive cells, and Ki67+ cells in the choroid in unaffected regions were calculated in an identical fashion.

### Statistical Analyses

Data analyses were performed using Microsoft Excel and GraphPad. Linear mixed-effects (LME) models were used to examine changes in wound healing attributable to use LKI, while generalized linear models (GLM) were used to examine differences in cell proliferation. To account for minor variations in wound healing assays, baseline wound area values were included in LME models as predictors of subsequent area values, and robust standard error terms were used to account for changes in variance observed over time (i.e. repeated measures). Wound healing was measured as the change in area from the start of the experiments within each well; values shown in figures are transformed to express raw area values as percentages of the baseline area. In cell proliferation GLM models, the ratio of EdU expression to DAPI expression was examined (truncated to 1), requiring the use of binomial models with logit linking functions and robust standard errors to evaluate differences in expression across set distances (Scratch, Proximal, and Distal). Post-hoc tests of groups differences per experiment were adjusted for multiple comparisons using the Holm-Bonferroni correction method. Group differences are reported as mean±standard error.

For in vivo analyses, descriptive statistics using mean±standard deviation (SD) were used. Student's t-test was used to compare parametric data between two groups with an equal variance. P<0.05 was considered statistically significant.

### Example 2

### LATS Kinase Inhibitors Promote Retinal Repair and Regeneration In Vitro and In Vivo

### Effect of Lats Kinase Inhibition on Wound Healing in RPE Monolayers In Vitro

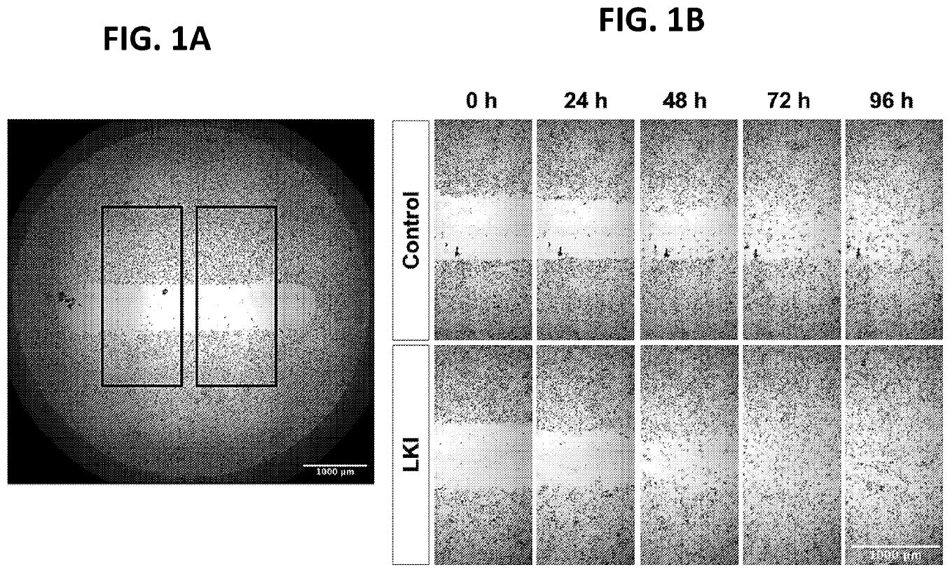

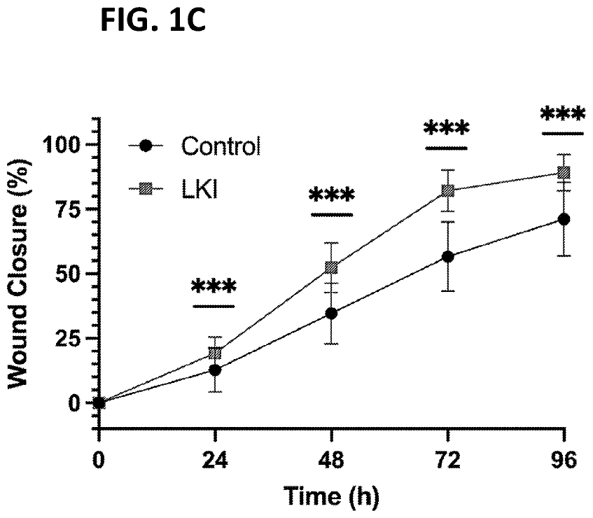

A wound healing assay, which involves creating a uniform scratch on a confluent monolayer of cells, is a commonly used method to study response to injury in vitro. To assess whether Hippo pathway inhibition promotes RPE regeneration, a wound healing assay was performed on primary human fetal RPE monolayers grown on 96-well plates in various culture conditions. RPE monolayers were subjected to a horizontal scratch and incubated with EdU and either LKI or vehicle. Wound closure was monitored every 24 hours for 96 hours total (FIGS. 1A and 1B).

In FBS-free media, there was significantly improved wound closure at all time points (24-96 hours) with LKI (n=27 regions for each time point) compared to control (n=30 regions for each time point) in primary human fetal RPE monolayers (FIGS. 1B and 1C). Three independent experiments were performed. The increase in wound closure with LKI compared to control was most pronounced at 48- and 72-hour time points. At 24 hours, the difference in wound closure was 8.25±1.49% (P<0.0001). At 48 and 72 hours, the difference in wound closure was 17.53±2.00% (P<0.0001) and 19.78±1.81% (P<0.0001), respectively. At 96 hours, the difference was 14.02±1.85% (P<0.0001).

Similarly, in FBS-containing media, there was significantly increased wound closure at all timepoints with LKI (n=13 regions) compared to DMSO (n=20 regions) in primary human fetal RPE monolayers, with a more pronounced difference seen earlier.

### Effect of Lats Kinase Inhibition on Cell Proliferation in RPE Monolayers In Vitro

To assess the effect of Lats kinase inhibition on cell proliferation in the absence of injury, RPE monolayers were incubated with EdU for 96 hours with the addition of LKI or DMSO. Cell proliferation was quantified as the percentage of EdU+ cells within 3 randomly selected regions within each well. In FBS-free media, there were significantly more EdU+ cells with LKI (n=54 regions from 18 wells, triplicate) compared to DMSO (n=54 regions from 18 wells, triplicate). Similarly, in FBS-containing media, there were significantly more EdU+ cells with LKI compared to DMSO.

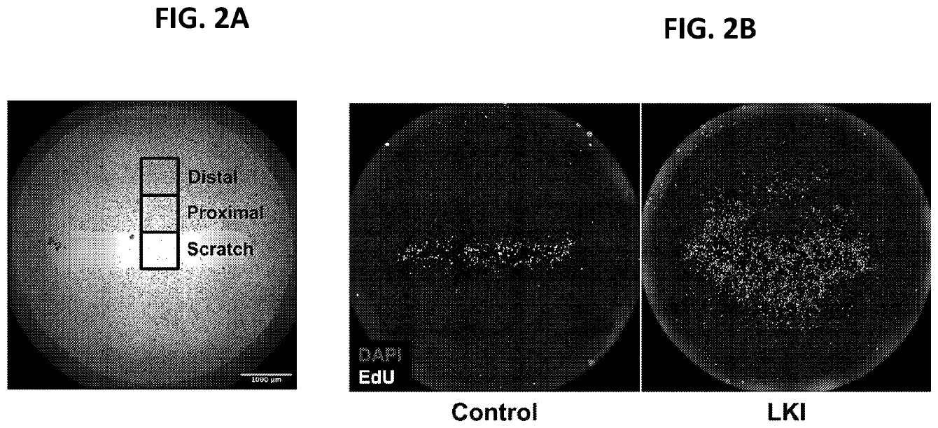

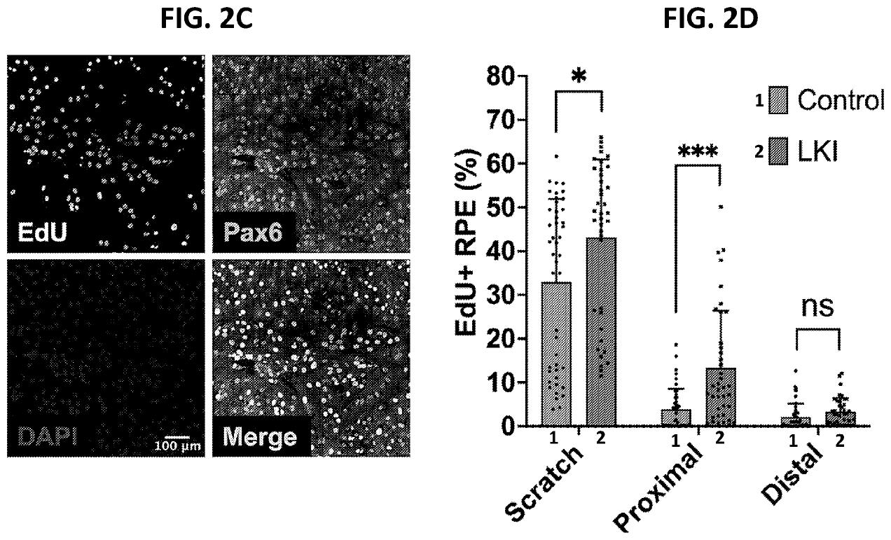

To assess whether Lats kinase inhibition accelerates wound healing via RPE proliferation in vitro, and the mechanism of wound closure with Lats kinase inhibition, RPE monolayers were incubated with EdU following the initial scratch and remained exposed to EdU for 96 hours. At 96 hours, cells were fixed and stained for various cell markers. The percentage of EdU+ cells within scratch, proximal, and distal regions was recorded (FIG. 2A).

There was a significant increase in the percentage of EdU+ cells in scratch and proximal regions but not distal regions with LKI (n=39 regions at each distance) compared to control (n=45 regions at each distance) (FIGS. 2B-2D). Three independent experiments were performed. In scratch regions, the percentage of EdU+ cells was 40.11±2.07% with LKI compared to 33.23±1.86% with control (difference 6.88±2.80%, P=0.04). At proximal regions, the percentage of EdU+ cells with LKI was 10.25±1.30% compared to 2.89±0.41% with control (difference 7.36±1.38%, P<0.0001). At distal regions, the percentage EdU+ cells was 3.20±0.48% with LKI versus 2.29±0.34% with control (difference 0.91±0.62%, P=0.14). These assays were performed using FBS-free media.

Similar to FBS-free media, there was a significantly higher percentage of EdU+ cells at the scratch site and proximal areas but not distal areas with LKI (n=21 regions at each distance) compared to LKI (n=15 regions at each distance) in FBS-containing media.

In all conditions, the percentage of EdU+ cells was greatest at the scratch site with significantly less in proximal areas. In distal areas, the percentages of EdU+ cells were similar to areas in wells that were not scratched.

Characterization of EdU+ RPE Cells with Lats Kinase Inhibition

In order to characterize RPE cell fate in response to Lats kinase inhibition in the context of injury, cells were stained with PAX6 and RPE65. PAX6 is a transcription factor that has been shown to regulate early differentiation of RPE cells by activating genes required for RPE cell function such as RPE65, a visual pigment cycling protein expressed in mature RPE.

In the experiments shown by FIGS. 2A-2D, at 96 hours post-scratch, nearly all EdU+ cells within the original scratch site expressed PAX6 in both LKI (99.67±1.46%) and control (99.94±0.26%) conditions (FIG. 2D). There was no significant difference in EdU/PAX6 doubly positive cells in the scratch site between LKI and control.

### Characterization of RPE Response to Injury In Vivo

To characterize the effect of laser injury on RPE in vivo, Dutch belted rabbits (n=2) received laser photocoagulation in a grid pattern with varying intensity (FIG. 3A). Following the laser procedure, fundus photos, infrared photos, and optical coherence tomography (OCT) imaging of the affected regions were obtained. To evaluate the effect of laser injury on RPE histologically, animals were sacrificed at 1- and 4-week time points following fundus and infrared photos and OCT imaging. H&E staining was performed.

As expected, higher intensity laser spots appeared bright and edematous on fundus photos following the laser procedure (FIG. 3B). After 1 week, laser spots appeared less bright and showed increased pigmentation on fundus photos. Lower intensity lesions were barely visible. After 4 weeks, the appearance on fundus photos had largely stabilized with slightly increased pigmentation compared to 1 week. Immediately following the laser procedure, there was intraretinal edema encompassing all layers, including the RPE, with higher intensity lesions resulting in increased edema (FIG. 3D). Laser spots appeared hyper-reflective compared to surrounding areas on OCT after 1 week (FIG. 3E). After 4 weeks, hypertransmission defects underlying laser spots were clearly seen (FIG. 3F).

Laser spots were clearly visible with H&E staining at both 1- and 4-week time points (FIGS. 3G and 3H). At 1 week, there were notable hyperplastic and hypertrophic changes to the RPE layer. Notable disruption to the laminar architecture of the retinal layers could be seen in all cases. In some cases, RPE cells could be seen within the outer and inner nuclear layer.

Given the appearance of laser spots on fundus photos, OCT, and histology, a power of 170 mW and duration 40 ms was selected for further assessment (FIGS. 31 and 3J). Higher intensity lesions severely disrupted the architecture of the retina and likely resulted in irreparable damage to the RPE layer. On the other hand, lower intensity likely did not damage the RPE sufficiently as no significant changes were seen on fundus photos or OCT after 4 weeks. Therefore, a middle intensity laser lesion was chosen (170 mW power and 40 ms).

### Effect of Lats Kinase Inhibition on RPE Response to Injury In Vivo

To evaluate the effect of Lats kinase inhibition on the RPE injury response in vivo, 17 Dutch-belted rabbits received photocoagulation procedure to cause focal disruptions of the RPE and photoreceptor layers in a 6×6 grid of laser spots. Following photocoagulation, each animal received an intravitreal injection of 50 μL of 15 mM LKI in the right eye (n=10 eyes) and 50 μL vehicle in the left eye (n=17 eyes). Fundus photos and OCT imaging were performed immediately following the laser procedure and at 1- and 4-week time points. Three independent experiments were performed.

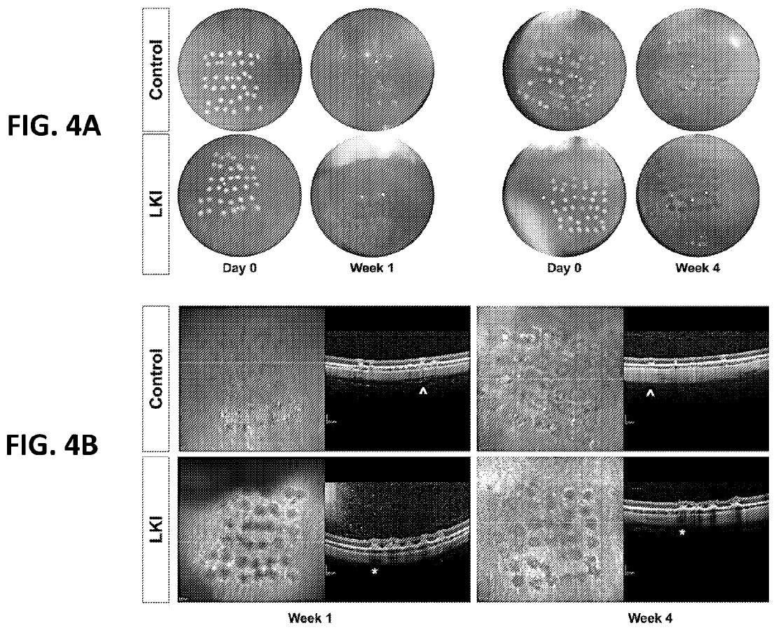

As expected, there was no difference in laser spot appearance on fundus photos or OCT in eyes injected with LKI compared to control immediately post-laser. After 1 week, lesions in eyes injected with LKI demonstrated increased pigmentation compared to control (FIG. 4A). On OCT, the RPE layer at the site of the laser spot appeared thicker with increased shadowing compared to control where hyper-transmission defects were apparent (FIG. 4B). At 4 weeks, lesions in eyes injected with LKI continued to demonstrate a robust increase in pigmentation on fundus photos compared to control (FIG. 4A). At the same time point, OCT demonstrated significantly increased thickness of the RPE layer in LKI compared to control and continued to show shadowing immediately below the RPE layer at the lesion sites (FIG. 4B). In contrast, the control eyes continued to demonstrate the hyper-transmission defects suggestive of persistent RPE loss.

### Effect of Lats Kinase Inhibition on Cell Proliferation In Vivo.

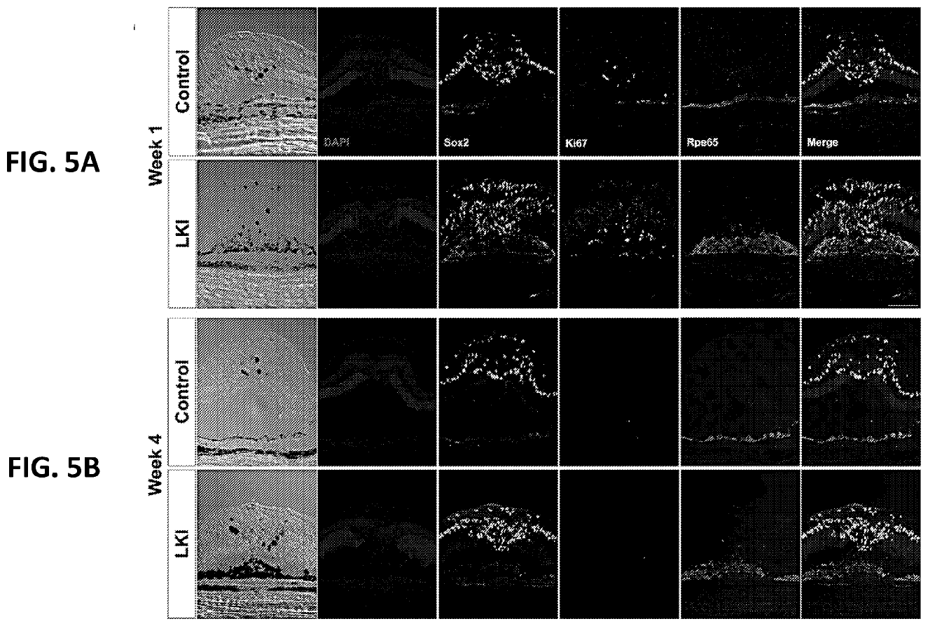

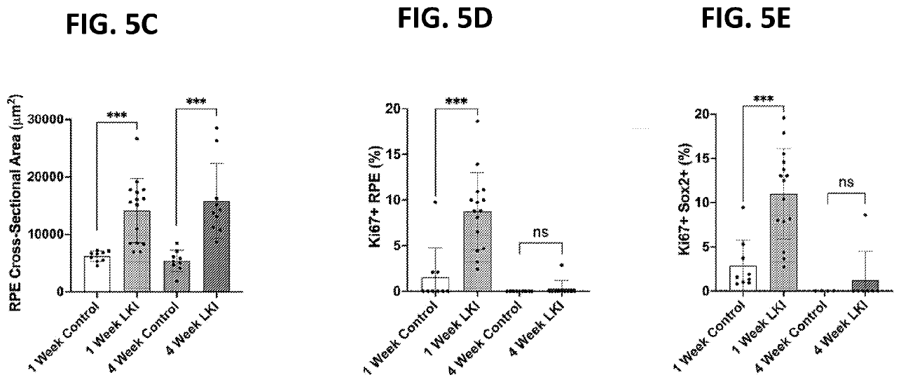

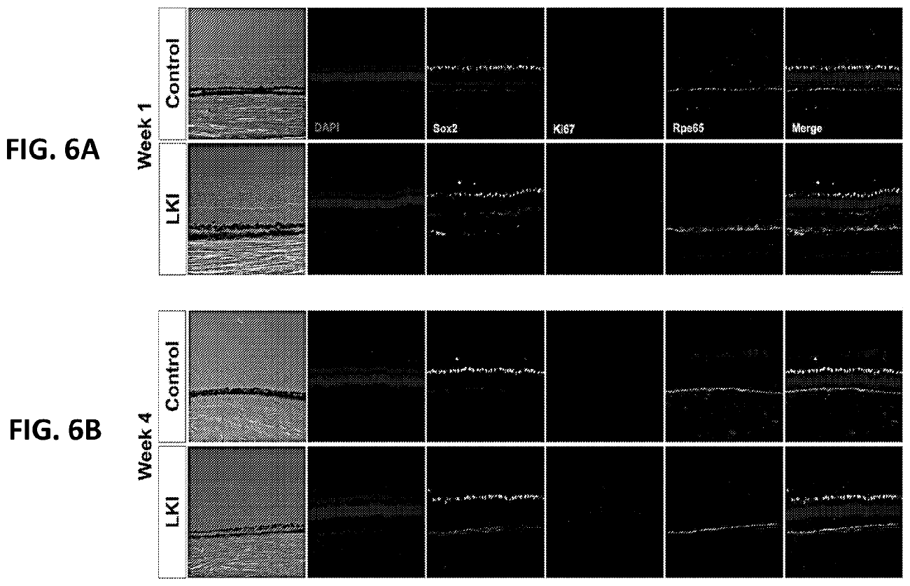

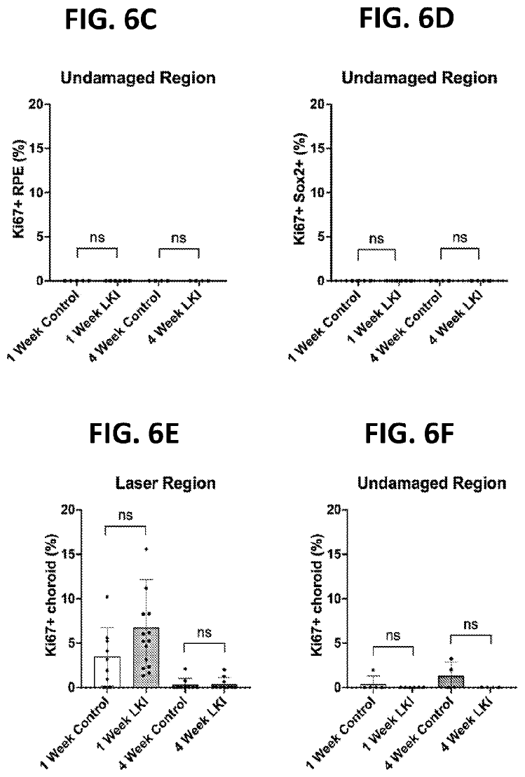

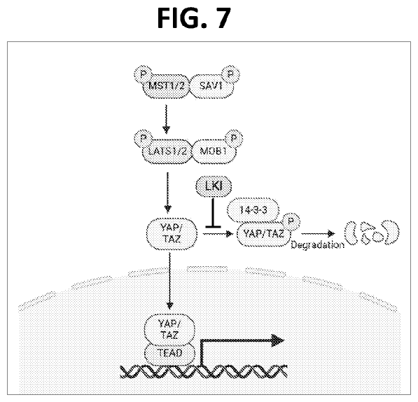

To further evaluate the effect of Lats kinase inhibition on the RPE injury response in vivo, eyes injected with LKI (n=9 eyes) or vehicle (n=8 eyes) from three independent experiments were cryosectioned and immunolabeled with Ki-67, a marker of cell proliferation. Additionally, sections were labeled with RPE65 to visualize RPE, Sox2 to visualize Müller glia, and Dapi to label all cell nuclei. After 1 week (FIG. 5A), laser spots in eyes injected with LKI (n=15 laser spots from 4 eyes) demonstrated a significant increase in underlying RPE cross-sectional area (FIG. 5C) and maximal thickness compared to control (n=9 laser spots from 4 eyes). RPE cross-sectional area was 14129±5605 μm² in LKI laser spots and 6221±914.2 μm² in control laser spots (P<0.0001). RPE maximal thickness was 56.36±19.23 μm in LKI laser spots and 23.08±8.46 μm in control laser spots (P<0.0001). Within the RPE layer, there was a significant increase in Ki67+ cells with LKI compared to the control (FIG. 5A, 5D). The percentage of Ki67+ RPE was 8.75±4.22% in LKI laser spots and 1.55±3.21% in control laser spots (P<0.001). Additionally, there was a significantly higher percentage of Sox2+Müller glia cells that were Ki67+(FIG. 5E). The percentage of Ki67+ Sox2+ cells was 11.00±5.11% in LKI laser spots and 2.89±2.88% in control laser spots (P<0.001). The effect was limited to the damaged RPE and Müller glia. There were no Ki67+ cells within the RPE layer or in Müller glia outside of laser spots after 1 week in eyes injected with LKI (n=6 laser spots from 3 eyes) or control (n=5 laser spots from 3 eyes). Within the choroid, there was also no significant difference in the number of Ki67+ cells in LKI laser spots (6.66±5.37%) or control laser spots (3.46±3.29%) (P=0.11) (FIGS. 6A-6F).

After 4 weeks (FIG. 5A), laser spots in eyes injected with LKI (n=10 laser spots from 5 eyes) showed increase in underlying RPE cross-sectional area and maximal thickness (FIG. 5C) compared to control (n=9 laser spots from 4 eyes). RPE cross-sectional area was 15803±6530 μm in LKI laser spots and 5396±18.58 μm² in control laser spots (P<0.001). At 4 weeks, there were very few Ki67+ cells within the RPE layer in LKI laser spots (0.29±0.90%) and no Ki67+ cells within the RPE layer in control laser spots. This difference was not significant (P=0.36) (FIG. 5D). The percentage of Ki67+ Sox2+ cells was 1.23±3.25% in LKI laser spots (n=7 laser spots from 3 eyes) and 0% in control laser spots (n=4 laser spots from 3 eyes), which was not significant (P=0.48) (FIG. 5E). Three LKI laser spots and five control laser spots were excluded from Sox2 analysis due to poor Sox2 staining. Similar to 1 week, there was no significant difference in the number of Ki67+ cells within the choroid in LKI laser spots (0.39±0.71%) or control laser spots (0.32±0.71) (P=0.82) or in undamaged regions (FIGS. 6A-6F).

### Example 3

### Functional Testing and Identification of Effective Dose

### Functional Testing

The laser photocoagulation model produces localized injury that does not cause measurable declines in vision. To test the functional effects of LKI-induced retinal regeneration in vivo, the well-established sodium iodate (SI) model of acute retinal pigment epithelium (RPE) injury can be used. Systemic or intravitreal SI has been shown to cause death of outer retinal cells in several mammalian models by inducing oxidative stress in the RPE. With RPE loss, the photoreceptor cells also degenerate, and this leads to permanent vision loss. The pathophysiology of SI injury is reminiscent of geographic atrophy in dry age-related macular degeneration (AMD), but also of other genetic and non-genetic retinal disorders affecting the RPE and photoreceptor cells. The SI model has several advantages that make it ideal to test the efficacy of LKI in retinal regeneration: 1) it induces reproducible acute injury to the RPE with secondary photoreceptor degeneration reminiscent of AMD; 2) the effect is titratable in order to mimic both early and late-stage disease; 3) the onset of damage is highly reproducible allowing us to determine the therapeutic window for LKI administration.

The protocol for SI retinal injury in mice has been described previously. Briefly, SI (NalO₃ S4007; Sigma-Aldrich) will be freshly dissolved in sterile PBS and injected intraperitoneally into male and female wild-type mice. For systemic injections, 10-30 mg/kg doses of SI were shown to generate moderate to severe RPE degeneration with secondary photoreceptor loss evident by 1-4 weeks after administration. Alternatively, an intravitreal injection of 1 μL of SI (1-10 mg/mL) will be used to induce a comparable level of degeneration.

Prior to or following injury, animals will be treated with a therapeutic dose of LKI or vehicle control. At some time points (e.g., e days, 1 week, and 4 weeks) after SI administration, RPE and photoreceptor damage will be assessed in treated and control groups in vivo using optical coherence tomography (OCT), autofluorescence, electroretinography, and/or visual behavior testing. The latter may include quantitative optomotor responses to functionally assess for differences in visual function in control and LKI-treatment animals. Once testing and imaging have occurred, animals will be sacrificed for immunohistochemistry to assess for anatomic corelates of functional outcomes. The main outcome of LKI administration is expected to be the restoration of the RPE layer, which will in turn prevent degeneration of the surviving photoreceptors. In addition, an LKI-mediated Müller glia (MG) proliferation was observed in the laser injury model. Because lower vertebrates can restore photoreceptors via proliferation and differentiation of Müller glia, it is also expected that de novo photoreceptor generation will be observed in the LKI-treated animals. Other models of retinal degeneration can also be used, such as the Royal College of Surgeons rat or the Merik knockout mouse.

### Identification of the Effective Dose

Using the laser injury model in rabbits we demonstrated that intravitreal administration of 50 μL of 15 mM LKI microsuspension induces extensive RPE proliferation and wound healing without measurable effects on off-target cell types or uninjured regions. Nevertheless, because the drug is highly insoluble, this dose of LKI microsuspension creates a depot that may stay in the eye for up to a month. Accordingly, we anticipate this dose to be an upper limit of the therapeutically effective range to be used in pre-clinical functional testing in mice (above) and clinical testing in humans. To determine the minimal effective dose and the duration of drug exposure in vivo, we will first perform a PK study in the rabbit model. Intravitreal injection of 50 μL LKI microsuspension of three concentrations (e.g., 15 mM, 5 mM, and 1.5 mM), each decreasing by a factor of about three, will be administered intravitreally into rabbit eyes. Following a single injection, soluble fraction of LKI will be measured via LC-MS in the neurosensory retina, the RPE/choroid, and in the plasma at e.g., 4 hours, 24 hours, 3 days, 10 days, and 28 days. 0.5-3 μM concentration of LKI should be maintained in the eye tissue for 3-5 days to elicit the therapeutic effect (e.g. Yap dephosphorylation, RPE and Miller glia proliferation).

The volume of the vitreous cavity in rabbit is approximately 1.5 ml. The identified dose in the rabbit will then be scaled accordingly when translated to other species, including the mouse (vitreous volume=10-15 μL) and humans (vitreous volume=4.5 mL).

### DISCUSSION

Effects of novel Lats kinase inhibitors, such as TDI-011536, on RPE have been evaluated in both in vitro and in vivo injury models.

In an in vitro wound healing assay, RPE cells may either migrate or proliferate to fill the gap created by the wound. A large increase in the percentage of EdU+ cells within the original scratch area and an even larger increase in areas immediately adjacent to the scratch were observed. Importantly, there was no significant difference in the rate of RPE proliferation further from the injury. An increase in the rate of wound closure was observed as early as 24 hours following the injury. This increase in wound closure was greatest after 48-72 hours and was still observed at 96 hours after injury. These findings suggest the effects of Hippo pathway inhibition via LKI are relatively quick-onset and restricted to an injury context, and the increased rate of wound closure observed in RPE monolayers exposed to the LKI is, at least in part, the result of increased cell proliferation stimulated by the compound specifically in the injured area. Furthermore, nearly all EdU+ cells within the original scratch region expressed Pax6, suggesting these proliferative cells differentiate into mature RPE.

Some of the key signals the Hippo signaling pathway responds to are cell density and contact inhibition. When cell-cell contact is suddenly lost, as occurs in a wound healing assay, the Hippo pathway is inhibited and cells alter gene expression to favor cell proliferation. Further inhibiting the Hippo pathway pharmacologically likely potentiates this effect and therefore results in an even more robust proliferative response. Indeed, the largest increase in percentage of EdU+ cells with LKI was seen in proximal regions, indicating that these cells are most responsive to changes in the microenvironment. The increase in EdU+ cells observed within the original scratch area likely represents progeny of proliferative cells in the proximal regions as well as migratory cells that underwent cell division within the scratch area.

Uncontrolled cell proliferation is one of the major hallmarks of cancer and a major concern when altering cell pathways involved in its regulation. Indeed, the Hippo pathway has been shown to play an important role in a variety of cancers, including liver, breast, and lung, among others. Therefore, it is important to consider the effects of Lats kinase inhibition in this context. In this study, we observed no difference in the percentage of EdU+ cells in regions not immediately adjacent to the wound. This suggests the proliferative effect seen in RPE with the LKI is specific to injury. In other words, Hippo pathway inhibition alone is not sufficient to induce proliferation outside of an injury context. Additionally, there was only a modest increase in the percentage of EdU+ cells in monolayers not subjected to injury, further supporting its specificity. Moreover, we observed the hyperpigmentation seen on fundus photos to be restricted to the initial laser spot area. No significant increase in hyperpigmentation was seen outside the initial spot area. OCT changes were likewise restricted to spot areas.

In the present in vitro injury model, nearly all RPE cells expressed both early and late markers of RPE differentiation after exposure to TDI-011536. This suggests that both migratory and proliferative RPE cells differentiate into mature RPE in response to Hippo pathway inhibition.

Like all cells, RPE cells respond to stress or injury via one or more adaptive mechanisms. In adults, the RPE is known to have limited regenerative potential. In some cases, RPE cells may slowly degenerate and result in geographic atrophy, as in dry AMD. In other cases, such as in proliferative vitreoretinopathy (PVR), RPE cells may proliferate and migrate to damaged regions of the retina, resulting in fibrosis. This process, termed epithelial-mesenchymal transition (EMT), is characterized by downregulation of ZO-1 and E-cadherin, among others, as well as upregulation in vimentin and N-cadherin. In both, the structure and function of RPE remains impaired and results in permanent vision loss.

Retinal laser photocoagulation is a commonly performed procedure that induces damage to outer retinal structures via thermal energy. Importantly, laser photocoagulation has been shown to result in characteristic loss of the RPE monolayer and degeneration of the overlying photoreceptor cells. Photocoagulation therefore serves as an acute model for geographic atrophy in dry AMD.

In the present study, numerous changes in RPE morphology suggestive of RPE regeneration following intravitreal LKI in an in vivo laser injury model were observed. After 1 week, laser spots in eyes injected with intravitreal LKI demonstrated increased pigmentation that persisted four weeks later. Conversely, control spots demonstrated central hyperpigmentation with surrounding hypopigmentation, suggesting RPE loss. Hyper-transmission defects appear as bright regions on OCT due to increased light transmission into the choroid when the RPE layer is attenuated or absent. These defects are associated with RPE loss seen in dry AMD. Indeed, hyper-transmission defects were common on OCT at both 1- and 4-week time points at the site of injury in control eyes. In eyes injected with LKI, however, increased light absorption from an increased thickness of the pigmented RPE layer resulted in shadowing below the laser spots. The notable increase in OCT shadowing at 4 weeks in eyes injected with LKI suggests that there were significant hyperplastic and/or hypertrophic changes in the RPE layer obstructing the OCT beam, consistent with the increased pigmentation on fundus photos. Hyper-reflective intraretinal foci may be seen as well, possibly representing migratory RPE within other nuclear layers.

The robust increase in RPE cross-sectional area at both 1- and 4-week time points seen with LKI is consistent with the in vitro results. Given the significant increase in Ki67+ cells within the RPE layer 1 week after the laser injury with LKI, this increase in RPE thickness is likely the result of RPE proliferation. Furthermore, an increase in Sox2/Ki67 doubly positive Müller glia cells at the site of laser injury with LKI was observed, demonstrating that Hippo pathway inhibition also increases Müller glia proliferation.

Importantly, the increase in Ki67+ RPE and Müller glia was only seen 1 week after the laser injury. By 4 weeks, there were very few Ki67+ RPE in both LKI and control eyes, demonstrating that the proliferative effects of Hippo pathway inhibition are limited to the time the drug is present in the vitreous. Furthermore, even at 1 week the effect was restricted to the injury site. No Ki67+ cells within the RPE layer were observed, nor any Ki67/Sox2 doubly positive cells in undamaged regions of the retina. This has important clinical implications as uncontrolled RPE or Müller glia cell proliferation within the retina could result in abnormal growth and scarring, as occurs in diseases such as proliferative vitreoretinopathy.

Another potential concern of Hippo pathway inhibition is uncontrolled cell proliferation within the choroid, as occurs in choroidal neovascularization. As expected, more Ki67+ cells were seen within the choroid 1 week compared to 4 weeks after the injury. This likely represents a normal response to endothelial damage from the laser injury. Importantly, however, an increase in the percentage of Ki67+ cells within the choroid with LKI was not seen at either time point, suggesting that Hippo pathway inhibition does not result in increased proliferation within the choroid.

Hippo pathway inhibition has been shown to be associated with various cancers, most notably gastrointestinal cancers. As such, constitutive modulation of this pathway does carry an inherent risk of neoplasia. However, the present application has demonstrated that this risk is mitigated through targeted delivery of the LKI that results in transient inhibition of the pathway. Strikingly, the proliferative effects of LKI do not persist after the initial injury nor do they affect undamaged regions. Additionally, no toxic effects were observed in vitro or in vivo.

Dysfunction or loss of RPE is associated with various retinal degenerations, the most common being dry AMD. The present application has demonstrated that LKIs, such as TDI-011536, induce RPE regeneration limited to the injury site in both in vitro and in vivo models of injury, and therefore can be used to treat various retinal degenerations including AMD. No toxic effects were observed in vitro and, more importantly, intravitreal injection of this compound was well-tolerated in an animal model.

It will be apparent that the precise details of the methods or compositions described may be varied or modified without departing from the spirit of the described aspects of the disclosure. We claim all such modifications and variations that fall within the scope and spirit of the claims below.

1. A method of treating retinal degeneration, comprising administering a therapeutically effective amount of a LATS kinase inhibitor to a subject with retinal degeneration.

2. The method of claim 1, wherein the LATS kinase inhibitor is TDI-011536:

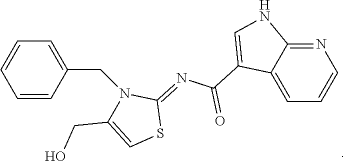 [SMILES: O=C(/N=c1\scc(CO)n1Cc1ccccc1)c1c[nH]c2ncccc12]

3. The method of claim 1, wherein the LATS kinase inhibitor is administered as an aqueous suspension formulation.

4. The method of claim 1, further comprising selecting the subject with retinal degeneration for treatment.

5. The method of claim 1, wherein the retinal degeneration is due to dry age-related macular degeneration (dry AMD), Stargardt disease, Best disease, myopic macular degeneration, commotio retinae (trauma), and/or an inherited retinal dystrophy.

6. The method of claim 5, wherein the retinal degeneration is due to dry age-related macular degeneration (dry AMD).

7. The method of claim 6, wherein the subject has early, intermediate, or late stage dry AMD.

8. The method of claim 7, wherein the subject has late stage dry AMD.

9. The method of claim 1, wherein the retinal degeneration is not due to
proliferation of retinal pigment epithelium (RPE) cells, or
wet age-related macular degeneration (wet AMD).

10. (canceled)

11. The method of claim 1, wherein the LATS kinase inhibitor is administered by intravitreal injection to the eye.

12. The method of claim 1, wherein the therapeutically effective amount of the LATS kinase inhibitor is administered in no more than five doses to treat the subject.

13. The method of claim 12, wherein the therapeutically effective amount of the LATS kinase inhibitor is administered in a single dose to treat the retinal degeneration in the subject.

14. The method of claim 11, wherein the LATS kinase inhibitor is administered by intravitreal injection to the eye at a dose of
about 10 μl to about 200 μl of about 5 to about 50 mM TDI-011536;
about 50 μl of about 5 to about 50 mM TDI-011536;
about 50 μl of about 15 mM TDI-011536; or
about 50 μl of about 45 mM TDI-011536.

15-17. (canceled)

18. The method of claim 1, wherein the therapeutically effective amount of the LATS kinase inhibitor is
about 0.1 to about 2.25 μmol, or
an amount sufficient to maintain about 0.5 to about 5 μM of the LATS kinase inhibitor in the neuroretina of the subject for about 3 to about 15 days following administration.

19. (canceled)

20. The method of claim 1, wherein treating the retinal degeneration in the subject delays progression of the retinal degeneration in the subject compared to a control.

21. The method of claim 20, wherein the subject has dry AMD and treating the retinal degeneration delays progression of dry AMD in the subject from early to intermediate stage dry AMD, or from intermediate to late stage dry AMD, by at least one year compared to a control.

22. (canceled)

23. The method of claim 1, wherein the subject has dry AMD and treating the retinal degeneration in the subject reduces the number and/or size of drusen under the retina in the subject.

24. The method of claim 1, wherein administering a dose of the LATS kinase inhibitor to the subject induces a short-term increase in proliferation of retinal pigment epithelium (RPE) cells and/or Müller glia cells in sites of retinal degeneration.

25-28. (canceled)

29. An in vitro method of identifying an agent for treating retinal degeneration, comprising:
providing a monolayer of retinal pigment epithelium (RPE) cells grown in tissue culture;
scraping the monolayer of RPE cells to produce a scraped RPE cell monolayer containing an RPE cell-free area within the monolayer;
incubating the scraped RPE cell monolayer with a test agent;
measuring proliferation of RPE cells in the scraped RPE cell monolayer in the presence of the test agent;
comparing the proliferation of RPE cells in the RPE cell-free area with a control, wherein the control represents proliferation of RPE cells in the RPE cell-free area when the scraped RPE cell monolayer is incubated in the presence of the LATS kinase inhibitor TDI-011536; and
identifying the test agent as an agent for treating retinal degeneration if proliferation of RPE cells in the RPE cell-free area in the presence of the test agent is greater than proliferation of RPE cells in the RPE cell-free area in the presence of TDI-011536; or
identifying the test agent as not an agent for treating retinal degeneration if proliferation of RPE cells in the RPE cell-free area in the presence of the test agent is not greater than proliferation of RPE cells in the RPE cell-free area in the presence of TDI-011536.

30-31. (canceled)

32. An in vivo method of identifying an agent for treating retinal degeneration, comprising:
providing a vertebrate animal model of retinal degeneration in a human, wherein an eye of the animal has an area of damaged retinal pigment epithelium;
administering a test agent to the eye of the animal model;
measuring proliferation of RPE cells in the damaged area of the retinal pigment epithelium following administration of the test agent;
comparing the proliferation of RPE cells in the damaged area of the retinal pigment epithelium with a control, wherein the control represents proliferation of RPE cells in the damaged area of the retinal pigment epithelium following administration of a therapeutically effective amount of the LATS kinase inhibitor TDI-011536 to the eye of the animal model; and
identifying the test agent as an agent for treating retinal degeneration if proliferation of RPE cells in the area of the damaged retinal pigment epithelium following administration of the test agent is greater than proliferation of RPE cells in the damaged area of the retinal pigment epithelium following administration of the LATS kinase inhibitor TDI-011536; or
identifying the test agent as not an agent for treating retinal degeneration if proliferation of RPE cells in the area of the damaged retinal pigment epithelium following administration of the test agent is not greater than proliferation of RPE cells in the damaged area of the retinal pigment epithelium following administration of the LATS kinase inhibitor TDI-011536.

33-35. (canceled)
